(19) United States
(12) Patent Application Publication
(10) Pub. No.: US 2026/0226079 A1
(43) Pub. Date: Aug. 6, 2026

(54) A STRATEGY FOR THE SELECTIVE REMOVAL OF MRI CONTRAST AGENTS

(71) Applicant: Portland State University, Portland, OR (US)

(72) Inventors: Mark Woods, Portland, OR (US); Karley B. Maier, Portland, OR (US)

(73) Assignee: Portland State University, Portland, OR (US)

(21) Appl. No.: 19/149,446

(22) PCT Filed: Jan. 23, 2024
(86) PCT No.: PCT/US2024/012589

§ 371 Date: Jul. 18, 2025

Related U.S. Application Data
(60) Provisional application No. 63/481,216, filed on Jan. 24, 2023.

(51) Int. Cl. C07F 5/00

(52) U.S. Cl. CPC C07F 5/003

(57) ABSTRACT

Disclosed herein is a method for removing contract agents, such as a gadolinium-based contrast agent, from a liquid. The contract agent may comprise a bioorthogonal reaction pair moiety and the method may comprise contacting a solid support that comprises a complementary bioorthogonal reaction pair moiety with the liquid. In some embodiments, the liquid comprises urine or blood.

### CROSS REFERENCE TO RELATED APPLICATION

This application claims the benefit of the earlier filing date of U.S. provisional patent application No. 63/481,216, filed Jan. 24, 2023, which is incorporated herein by reference in its entirety.

### ACKNOWLEDGMENT OF GOVERNMENT SUPPORT

This invention was made with government support under Grant No. 1R21GM127964 awarded by the National Institutes of Health, and Grant No. 1638278 awarded by the National Science Foundation. The government has certain rights in the invention.

### FIELD

Disclosed herein are embodiments of compounds useful as contrast agents, and embodiments of a method for removing a contrast agent from a liquid.

### BACKGROUND

Clinical introduction of gadolinium-based contrast agents (GBCAs) for contrast-enhanced magnetic resonance imaging (MRI) has proven to be an invaluable diagnostic tool that has saved many lives. However, the long-term effects of gadolinium pollution from decades of unmitigated MRI contrast agent release into freshwater and seawater are beginning to unfold. Numerous aquatic species have suffered negative impacts from increasing concentrations of Gd³⁺, and the effect of bioaccumulation and direct exposure will likely impact human health, and there have been calls for public policy changes to address Gd³⁺ pollution. It is has become clear that there is a pressing need to develop a strategy for GBCA interception to prevent further environmental release of Gd³⁺.

### SUMMARY

Disclosed herein are embodiments of a method for removing contrast agents, such as gadolinium-based contrast agents, from a liquid, for example, urine or blood from a subject that has been administered the contrast agent. In some embodiments, the method comprises contacting a solid support comprising a first bioorthogonal reaction pair moiety with a liquid comprising a contrast agent comprising a second bioorthogonal reaction pair moiety that is complementary to the first bioorthogonal reaction pair moiety. The contrast agent may be a metal chelate where the metal is gadolinium(III), manganese(II) or iron(III). In certain embodiments, the contrast agent is a gadolinium-based contrast agent.

In some embodiments, the contrast agent comprises a ligand moiety and a metal ion where the ligand moiety is selected from any one of Formulas I-A to IV-C:

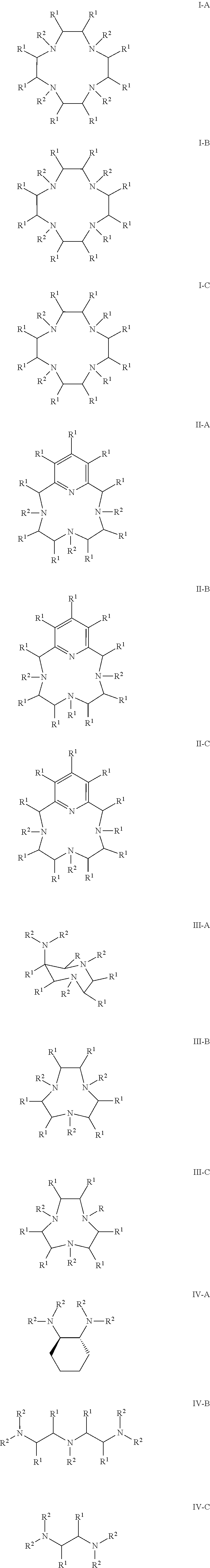

wherein each R¹ independently is R or —L—BG, where at least one R¹ is —L—BG; each R independently is H, aliphatic, or aryl; L is a bond, or a linker, such as —C₁₋₆alkyl-, —C₁₋₆alkyl-C(O)—, —O—C₁₋₆alkyl-, —O—C₁₋₆alkyl-C(O)—, —NHC₁₋₆alkyl-, or —NHC₁₋₆alkylC(O)—; BG is the second bioorthogonal reaction pair moiety; and R² is selected from

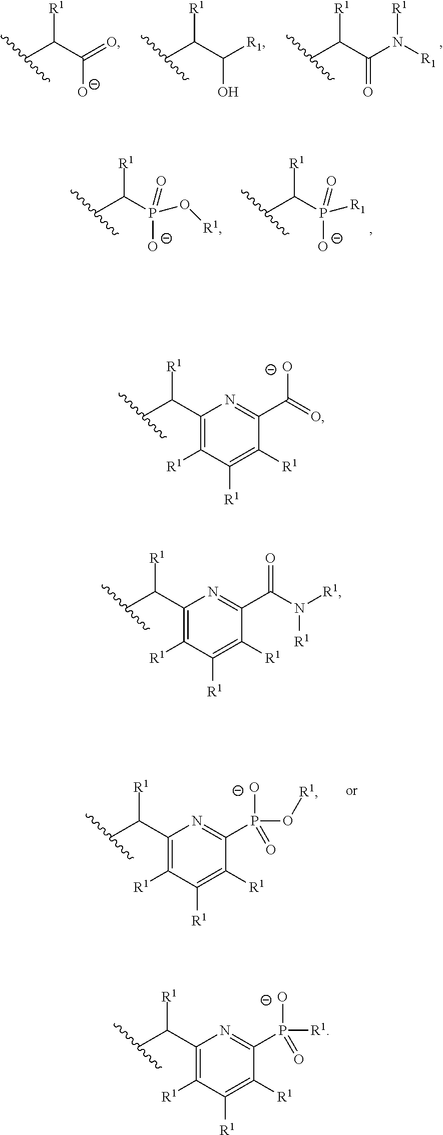

And in certain embodiment, the metal ion in the contrast agent is selected from Gd³⁺, Mn²⁺ or Fe³⁺, and may be Gd³⁺.

In some embodiments, the contrast agent has a structure according to any one of Formulas V-A to VIII-C

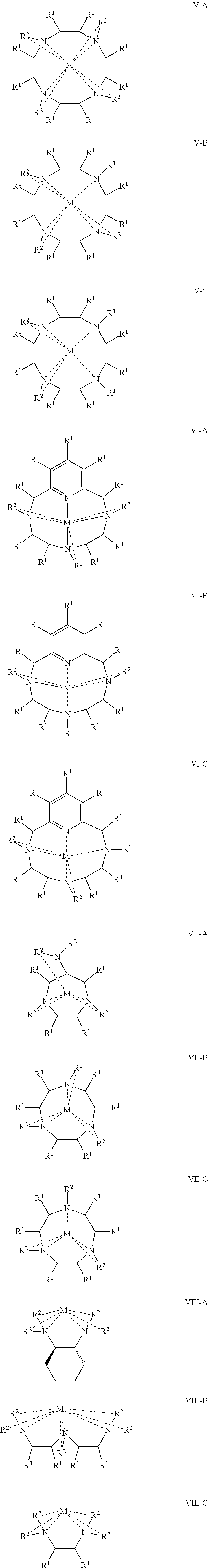

With respect to Formulas V-A to VIII-C, each R¹ independently is R or —L—BG, where at least one R¹ is —L—BG. Each R independently is H, aliphatic, or aryl. L is a bond, —C₁₋₆alkyl-, —C₁₋₆alkyl-C(O)—, —O—C₁₋₆alkyl-, —O—C₃₋₆alkyl-C(O)—, —NHC₃₋₆alkyl-, or —NHC₁₋₆alkylC(O)—, and BG is the second bioorthogonal reaction pair moiety. R² is selected from

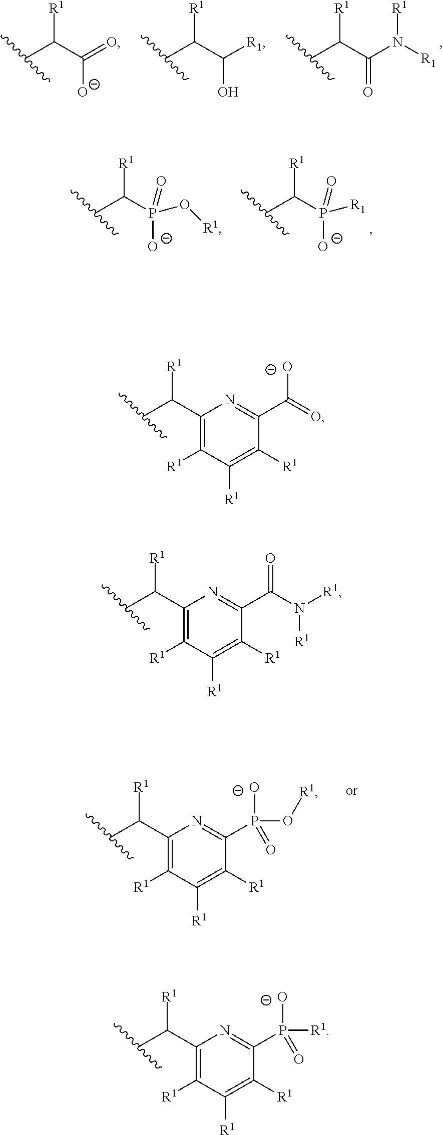

And M is selected from Gd³⁺, Mn²⁺ or Fe³⁺.

Also with respect to Formulas V-A to VIII-C, dotted lines “ [symbol] ” indicate an optional bond from the metal M to a nitrogen atom; dashed lines “ [symbol] ” indicate one or two optional bonds from the metal M to an R² substituent; and the contrast agent comprises at least three of the optional dotted and/or dashed bonds, such as at least four, at least five, at least six, or at least seven of the optional dotted and/or dashed bonds.

In some embodiments, M is Gd³⁺ and the contrast agent comprises at least seven of the optional dotted and/or dashed bonds to the metal.

In other embodiments, M is Fe³⁺ or Mn²⁺, and the contrast agent comprises at least five of the optional dotted and/or dashed bonds to the metal.

In certain embodiments, the contrast agent has a structure according to any one of Formulas V-A, V-B, V-C, VI-A, VI-B or VI-C, and at least three of the dotted bonds between the metal and a nitrogen are present, and at least two of the dashed bonds between the metal and an R² substituent are present.

In other particular embodiments, the contrast agent has a structure according to any one of Formulas VII-B or VII-C, and at least three dotted bonds between the metal and a nitrogen are present. But in alternative embodiments, the contrast agent has a structure according to Formula VII-B, M is Fe³⁺ or Mn²⁺, and no dotted bonds between the metal and nitrogen are present, but each of the dashed bonds between the metal and the R² substituents are present, indicating the presence of one or two bonds between M and R², depending on the structure of R².

In particular embodiments, the contrast agent has a structure according to Formula IX or Formula X

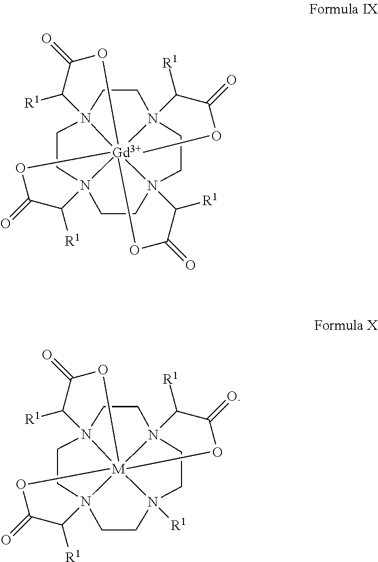

With respect to Formulas IX and X, R¹ is as previously defined for Formulas I-A to IV-C, and M is Mn²⁺ or Fe³⁺

In any embodiments, the first and second bioorthogonal reaction pairs, in any order, may be, but are not limited to,

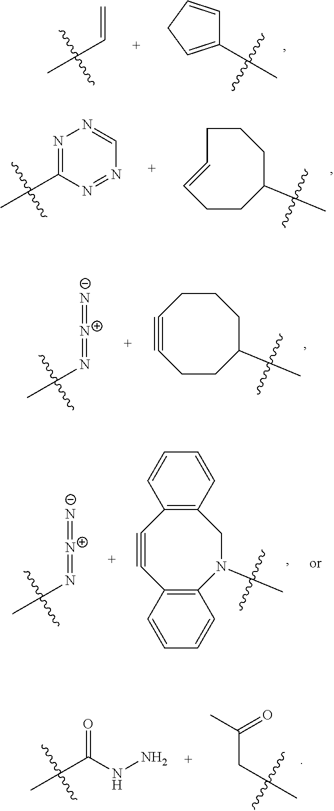

And in any embodiments, the solid support may be any solid support suitable for use in the disclosed method, such as, but not limited to, silica, peptide resin, or polymeric beads. In one embodiment, the solid support is or comprises polystyrene beads.

Also disclosed herein are embodiments of a contrast agent having a structure disclosed herein.

The foregoing and other objects, features, and advantages of the invention will become more apparent from the following detailed description, which proceeds with reference to the accompanying figures.

### BRIEF DESCRIPTION OF THE DRAWINGS

FIG. 1 is a schematic diagram illustrating how the GBCAs functionalized with a bioorthogonal moiety (blue arrows) and subsequent selective interception onto solid support bead functionalized with the complementary bioorthogonal reaction partner (black chevrons).

FIG. 2 is a reaction scheme illustrating an exemplary synthetic pathway for the bioorthogonal reactions disclosed herein.

FIG. 3 is a reaction scheme illustrating the synthetic strategy for 3-azidopropan-1-amine and subsequent functionalization through onto carboxypolystyrene through activated coupling with EDC and NHS.

FIG. 4 is an FT-IR of azide functionalized carboxypolystyrene beads with highlighted characteristic N₃ stretch at 2096 cm⁻¹.

FIG. 5 is a reaction scheme illustrating the synthesis of alkyne-functionalized fluoroscein dye (top) coupled to azide terminated polystyrene beads through a Cu(I) catalyzed click reaction and resulting fluoroscein carboxystyrene beads.

FIG. 6 is a digital image illustrating the difference in appearance of the starting azide polystyrene beads (left) and the fluoroscein functionalized beads (right).

FIG. 7 is a reaction scheme illustrating the synthetic strategy for functionalization of carboxypolystyrene with DBCO amine accessed by methyl esterification and subsequent substitution for amide linkage formation.

FIG. 8 is an FT-IR spectra of neutralized carboxypolystyrene beads (top, blue trace) and an attempt of methyl esterification (bottom, red trace).

FIG. 9 is a reaction scheme that illustrates an exemplary synthetic route for an alkyne-functionalized GdTTHA chelate.

FIG. 10 is a reaction scheme illustrating exemplary synthetic routes employed for GdDO3A-DBCO monoamide.

FIG. 11 is a reaction scheme illustrating a synthetic scheme for chelation of LnDO3A-azido monoamide.

FIG. 12 provides the structures of reagents GdTTHA-Alkyne and azide polystyrene that were utilized for initial click experiments with CuSO4 and ascorbic acid reducing agent.

FIG. 13 provides the structures of reagents GdDO3A-DBCO monoamide and azide polystyrene utilized in initial SPAAC experiments for selective interception.

FIG. 14 is a graph of residual Gd³⁺ % versus time, illustrating the selective interception of Gd over time with GdDO3A-DBCO monoamide chelate and azide terminated PS beads (blue spheres) and simulated second order reaction with rate constant 48.4 M⁻¹s⁻¹.

FIG. 15 provides the structures of reagents GdDO3A-azido monoamide and DBCO polystyrene utilized in SPAAC experiments for selective interception.

FIG. 16 is a plot of Gd ligand versus DMSO concentration versus time, illustrating the comparison of % Gd remaining in a water matrix and the effect of % DMSO cosolvent, time and substitution strategy.

FIG. 17 is a plot of Gd ligand versus DMSO concentration versus time, illustrating the comparison of % Gd remaining in a Surine™ matrix and the effect of % DMSO cosolvent, time and substitution strategy.

FIG. 18 is a plot illustrating the GdDO3A-DBCO monoamide temperature-dependent R₂ₚ relaxation rate of ¹⁷O measured at 67.8 MHz (11.74 T) pH 6.8, and a Gd³⁺ concentration of 6.5 mM.

FIG. 19 is a plot illustrating the GdDO3A-DBCO monoamide ¹H NMRD profiles at 283 (blue, top line), 298 (black, middle line) and 310 K (red, lower line), measured at pH 6.8, and a Gd³⁺ concentration of 1.32 mM.

FIG. 20 is a plot illustrating the GdDO3A-azido monoamide temperature-dependent R₂ₚ relaxation rate of ¹⁷O measured at 67.8 MHz (11.74 T) pH 7.0, and a Gd³⁺ concentration of 8.5 mM.

FIG. 21 is a plot illustrating the GdDO3A-azido monoamide ¹H NMRD profiles at 283 (blue, top line), 298 (black, middle line) and 310 K (red, lower line), measured at pH 7.0.

FIG. 22 is a table of exemplary bioorthogonal reaction pair, illustrating how each pair of bioorthogonal reaction pairs combines together to link the two moieties represented by the two circles.

### DETAILED DESCRIPTION

### I. Definitions

The following explanations of terms and methods are provided to better describe the present disclosure and to guide those of ordinary skill in the art in the practice of the present disclosure. The singular forms “a,” “an,” and “the” refer to one or more than one, unless the context clearly dictates otherwise. The term “or” refers to a single element of stated alternative elements or a combination of two or more elements, unless the context clearly indicates otherwise. As used herein, “comprises” means “includes.” Thus, “comprising A or B,” means “including A, B, or A and B,” without excluding additional elements. All references, including patents and patent applications cited herein, are incorporated by reference.

Unless otherwise indicated, all numbers expressing quantities of components, molecular weights, percentages, temperatures, times, and so forth, as used in the specification or claims are to be understood as being modified by the term “about.” Accordingly, unless otherwise indicated, implicitly or explicitly, the numerical parameters set forth are approximations that may depend on the desired properties sought and/or limits of detection under standard test conditions/methods. When directly and explicitly distinguishing embodiments from discussed prior art, the embodiment numbers are not approximates unless the word “about” is recited.

Unless explained otherwise, all technical and scientific terms used herein have the same meaning as commonly understood to one of ordinary skill in the art to which this disclosure belongs. Although methods and materials similar or equivalent to those described herein can be used in the practice or testing of the present disclosure, suitable methods and materials are described below. The materials, methods, and examples are illustrative only and not intended to be limiting.

Aliphatic: A substantially hydrocarbon-based compound, or a radical thereof (e.g., C₆H₁₃, for a hexane radical), including alkanes, alkenes, alkynes, including cyclic versions thereof (also referred to as cycloaliphatic), and further including straight- and branched-chain arrangements, and all stereo and position isomers as well. Unless expressly stated otherwise, an aliphatic group contains from one to twenty-five carbon atoms; for example, from one to fifteen, from one to ten, from one to six, or from one to four carbon atoms. The term “lower aliphatic” refers to an aliphatic group containing from one to ten carbon atoms. An alkyl group is a hydrocarbon group having a saturated carbon chain. The chain may be cyclic, branched or unbranched. When the chain is cyclic, the group may be referred to as cycloalkyl. Examples, without limitation, of alkyl groups include methyl, ethyl, propyl, butyl, pentyl, hexyl, heptyl, octyl, nonyl and decyl. The term lower alkyl means the chain includes 1-10 carbon atoms. The terms alkenyl and alkynyl refer to hydrocarbon groups having carbon chains containing one or more double or triple bonds, respectively; cyclic versions may be referred to a cycloalkenyl or cycloalkynyl, respectively. An aliphatic chain may be substituted or unsubstituted. Unless expressly referred to as an “unsubstituted aliphatic,” an aliphatic group can either be unsubstituted or substituted. An aliphatic group can be substituted with one or more substituents (up to two substituents for each methylene carbon in an aliphatic chain, or up to one substituent for each carbon of a —C═C— double bond in an aliphatic chain, or up to one substituent for a carbon of a terminal methine group). Exemplary substituents include, but are not limited to, alkyl, alkenyl, alkynyl, alkoxy, alkylamino, alkylthio, acyl, aldehyde, amide, amino, aminoalkyl, aryl, arylalkyl, carboxyl, cyano, cycloalkyl, dialkylamino, halo, haloaliphatic, heteroaliphatic, heteroaryl, heterocycloaliphatic, hydroxyl, oxo, sulfonamide, sulfhydryl, thioalkoxy, or other functionality. A substituted aliphatic group includes at least one sp³-hybridized carbon or two sp²-hybridized carbons bonded with a double bond or at least two sp-hybridized carbons bonded with a triple bond.

Aromatic: Unsaturated, cyclic hydrocarbons having alternate single and double bonds. Benzene, a 6-carbon ring containing three double bonds, is a typical aromatic compound.

Aryl: A monovalent aromatic carbocyclic group of, unless specified otherwise, from 6 to 15 carbon atoms having a single ring (e.g., phenyl) or multiple condensed rings in which at least one ring is aromatic (e.g., quinoline, indole, benzodioxole, and the like), provided that the point of attachment is through an atom of an aromatic portion of the aryl group and the aromatic portion at the point of attachment contains only carbons in the aromatic ring. If any aromatic ring portion contains a heteroatom, the group is a heteroaryl and not an aryl. Unless expressly referred to as an “unsubstituted aryl,” an aryl group can either be unsubstituted or substituted.

Substituent: An atom or group of atoms that replaces another atom in a molecule as the result of a reaction. The term “substituent” typically refers to an atom or group of atoms that replaces a hydrogen atom, or two hydrogen atoms if the substituent is attached via a double bond, on a parent hydrocarbon chain or ring. The term “substituent” may also cover groups of atoms having multiple points of attachment to the molecule, e.g., the substituent replaces two or more hydrogen atoms on a parent hydrocarbon chain or ring. In such instances, the substituent, unless otherwise specified, may be attached in any spatial orientation to the parent hydrocarbon chain or ring. Exemplary substituents include, for instance, alkyl, alkenyl, alkynyl, alkoxy, alkylamino, alkylthio, acyl, aldehyde, amido, amino, aminoalkyl, aryl, arylalkyl, arylamino, carbonate, carboxyl, cyano, cycloalkyl, dialkylamino, halo, haloaliphatic (e.g., haloalkyl), haloalkoxy, heteroaliphatic, heteroaryl, heterocycloaliphatic, hydroxyl, oxo, sulfonamide, sulfhydryl, thio, and thioalkoxy groups.

Substituted: A fundamental compound, such as an aryl or aliphatic compound, or a radical thereof, having coupled thereto one or more substituents, each substituent typically replacing a hydrogen atom on the fundamental compound. A person of ordinary skill in the art will recognize that compounds disclosed herein may be described with reference to particular structures and substituents coupled to such structures, and that such structures and/or substituents also can be further substituted, unless expressly stated otherwise or context dictates otherwise. Solely by way of example and without limitation, a substituted aryl compound may have an aliphatic group coupled to the closed ring of the aryl base, such as with toluene. Again, solely by way of example and without limitation, a long-chain hydrocarbon may have a hydroxyl group bonded thereto.

### II. Overview

Typically, contrast agents (CAs), such as GBCAs, are not metabolized and are cleared, intact, through the renal system, with the first post-examination urine excretion containing up to 90% of the dose. A contrast agent may be based around any paramagnetic metal ion that has a sufficiently long electronic relaxation time constant. Without being bound to a particular theory, this may be achieved by having a metal ion with a symmetrical electronic structure that minimizes inter-electron interactions, for example, Gd³⁺, and high spin Mn²⁺ and Fe³⁺. Collection of the first urine pass after CA injection is therefore a viable approach to prevent CAs from entering wastewater treatment plants (WWTPs) where they are eventually released into surface waters and oceans. Urine collection bags to reduce X-ray contrast pollution have recently been implemented for a survey on patient compliance in Germany with optimistic responses. However, long-term storage of the urine sample is highly impractical. Moreover with respect to GBCAs, environmentally detrimental Gd mining could potentially be reduced through reclamation of Gd from urine samples.

Thus, disclosed herein are embodiments of an agent with the capability for selective removal from the first post-contrast excretion before the urine undergoes water treatment. Also disclosed herein is a strategy for selective interception of CAs from aqueous media through functionalization of the ligand scaffold.

Functionalized ligand scaffolds known as bifunctional chelators, BFCs, have been widely studied for diagnostic and therapeutic applications. For diagnostic imaging research, BFCs are most commonly employed as responsive or molecular imaging agents. However, the system disclosed herein is distinct from such uses. The functionalization strategy for selective ligand interception required a functionalized ligand scaffold containing a chemical moiety which remained intact through intravascular lifetime and renal excretion. The moiety must therefore impart a minimal effect on pharmacokinetic clearance, be unreactive towards endogenous biological functional groups, and have specific reactivity that would allow for post-contrast manipulation and extraction from a urine matrix. It was hypothesized that the reactivity characteristic of bioorthogonal chemical reaction pairs held the potential to meet these requisites. Therefore, bioorthogonal reaction partners were functionalized onto the ligand structure of CA-coordinating ligands to develop a system by which CAs can be selectively intercepted from the first post-contrast body clearance.

As indicated above, bioorthogonal reactions pairs are characterized by favorable reaction kinetics, high atom economy and, most importantly, reactivity that does not interfere or interact with native biological mechanisms. The term bioorthogonal chemistry was first described with respect to mucin-type O-linked glycoprotein roles in biological interactions through selective fluorescent tagging with bioorthogonal functional groups. This prompted the investigation and development of several new bioorthogonal reaction pairs which have been utilized in a wide range of applications from the original cellular mechanistic studies to drug delivery, genetic code expansion, and recently, in vivo pre-targeted imaging.

Additionally, the contrast agent and/or the gadolinium metal, may be recycled by the disclosed method. In some embodiments, the metal is released from the contrast agent by a suitable technique and can be recycled for further use in a contrast agent, for use in other applications, or for safe disposal. Suitable techniques include, but are not limited to, acid catalyzed dissociation, and/or combustion. Additionally, or alternatively, the contrast agent may be recycled for use or removed from the bioorthogonal reaction product for disposal.

### III. Design Concept for Selective Interception of CAs, Such as GBCAs

Disclosed herein is a method for using bioorthogonal reaction pairs for selective filtration of CAs. One partner of a bioorthogonal reaction pair is attached onto the ligand structure and the complementary partner is functionalized onto a solid support, such as silica, peptide resin, or polymeric bead, for example, a polystyrene (PS) bead. The combination of the bioorthogonal reaction pairs then selectively intercepts the ligand through filtration. To the inventors' knowledge, bioorthogonal chemistry has not previously been used for selective filtration in this way.

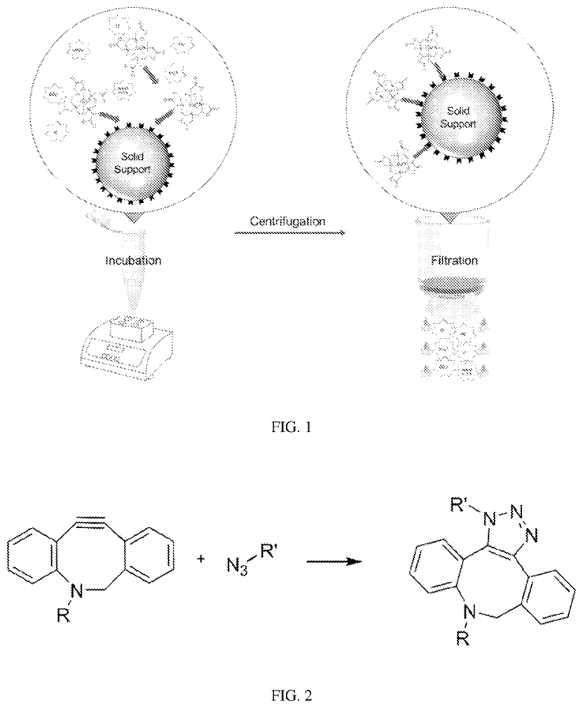

FIG. 1 is a schematic diagram illustration how the selective filtration of CAs is achieved. In FIG. 1 the bioorthogonal partner installed on the ligand (represented by blue arrows) remains intact through urine excretion. Upon elution through a solid support filter functionalized with the bioorthogonal reaction partner (black chevrons), the agent undergoes a chemoselective heterogeneous extraction from the liquid media, leaving all other matrix constituents to be flushed through the filter (to wastewater treatment). Ideally, the metal, such as Gd³⁺, in these complexes can be reclaimed and recycled to reduce further pollution from mining. The metal ion, such as Gd³⁺, or the CA can then be recycled and re-used as required.

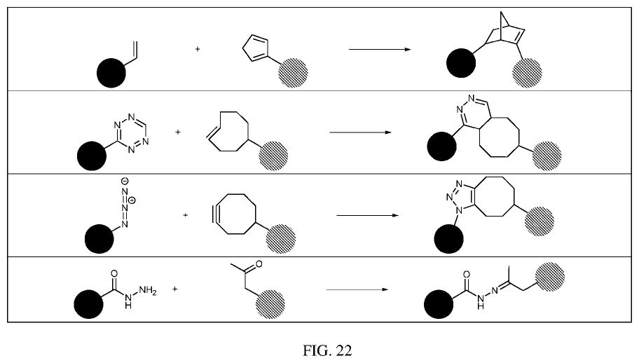

There are numerous bioorthogonal reaction pairs reported to date, most of which are well established and highly efficient. FIG. 22 provides a table illustrating several exemplary biorthogonal reaction pairs. A person of ordinary skill in the art understands that the reactive groups shown in FIG. 22 may be further substituted in such a way as to still allow the biorthogonal pairs to react. See, for example, FIG. 2 which demonstrates one example of how the triple bond-containing moiety may be substituted.

Selection of which bioorthogonal reaction pairs to survey for proof of principle of CA extraction involved consideration of a number of key factors. First, reaction partners which were synthetically accessible or commercially available, yet not prohibitively expensive, were identified.

Additionally, reactions were identified which had demonstrated a minimal amount of pharmacokinetic disruption to the biostructure on which they were functionalized. Ultimately, given these criteria, the azide moiety was selected due to its reported small effect on native activity and pharmacokinetics of biostructures in previous research efforts. The small, bioinert azide functional group undergoes 1,3 dipolar cyclization with terminal alkynes in a Cu(I)-catalyzed azide alkyne cycloaddition (CuAAC) (FIG. 2 top) as well as with ring strained cycloalkynes in the strain-promoted alkyne azide cycloaddition (SPAAC) (FIG. 2 bottom) to form a stable and inert triazole linkage.

Both CuAAC and SPAAC reactions display favorable kinetics with rate constants on the orders of 10-100 M⁻¹ s⁻¹ (20 μM Cu⁺) and 1-60 M⁻¹ s⁻¹, respectively. The CuAAC is not wholly bioorthogonal due to cytotoxicity of the copper(I) catalyst; however, the reagents were significantly less expensive and were utilized as a starting point in initial designs, characterization and synthetic strategies.

Due to synthetic accessibility, substitution of a CA scaffold with the ring strained dibenzylcyclooctyne (DBCO) was initially attempted, which would provide a useful chromatographic handle for purification. However, the possibility of distinct reactivity and efficiency was identified if the functionalization of the reaction partners on either bead or chelate were reversed. Therefore, synthetic strategies for functionalization of a CA with both reaction pairs were developed. The complementary solid support polystyrene microbeads were similarly functionalized with both DBCO and an azide functional group to demonstrate initial proof of principle for selective extraction from an aqueous medium. Finally, the studies were expanded to explore efficiency in the presence of variables such as pH, reaction time, temperature, solvent, and a simulated urine matrix to further characterize these systems.

### IV. Contrast Agent and Solid Support

The solid support may be any solid support suitable or use in the disclosed technology. In some embodiments, the solid support is or comprises silica, peptide resin, or polymeric beads, such as polystyrene beads. The solid support further comprises a first moiety of a biorthogonal reaction pair.

The CA may be functionalized with a second moiety of the biorthogonal reaction pair, such that the CA retains its effectiveness as a contrast agent while the second bioorthogonal reaction pair moiety is available to react with its complementary reaction pair, specifically, the first biorthogonal reaction pair moiety that is incorporated onto the solid support. In some embodiments, the first and second bioorthogonal reaction pair moieties are selected from, in any order:

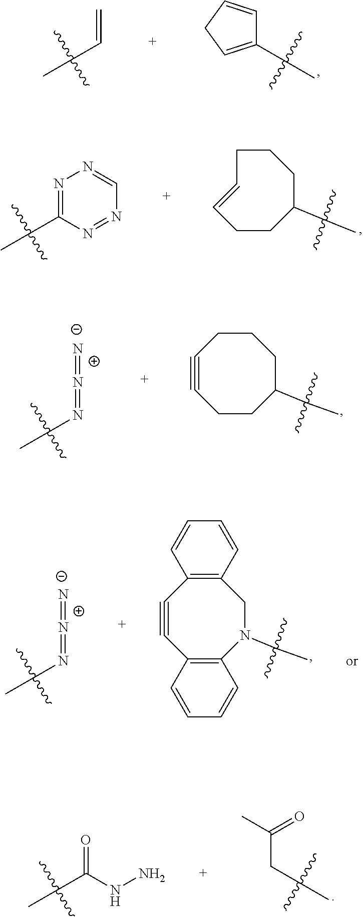

In some embodiments, the CA comprises a ligand having a structure selected from the Formulas I-A to IV-C:

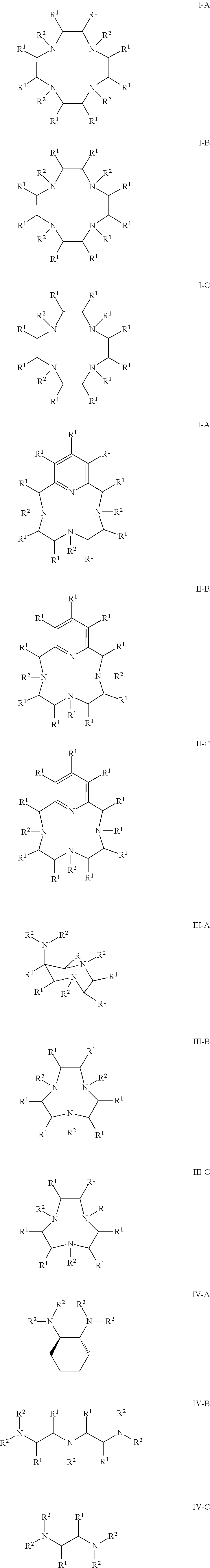

With respect to these formulas, each R¹ independently is R or —L—BG, where at least one R¹ is —L—BG. In some embodiments, exactly one R¹ is —L—BG.

Each R independently may be H or may be aliphatic or aryl, such as a C₁₋₆aliphatic or C₆₋₁₅aryl. In some embodiments, each R independently is an aryl or aliphatic group having an sp²-hybridized or sp-hybridized carbon at its attachment point to a carbon atom alpha to nitrogen. In some embodiments, each R independently is aryl, alkenyl, or alkynyl. One or more R groups may be phenyl, or substituted phenyl, for example, substituted with —CO₂H, or C₁₋₆alkyl.

In some embodiments, each R group present on the compound is the same, but in other embodiments, one or more R group is different from other R groups. In some embodiments, each R group is not H.

Each R² independently is selected from

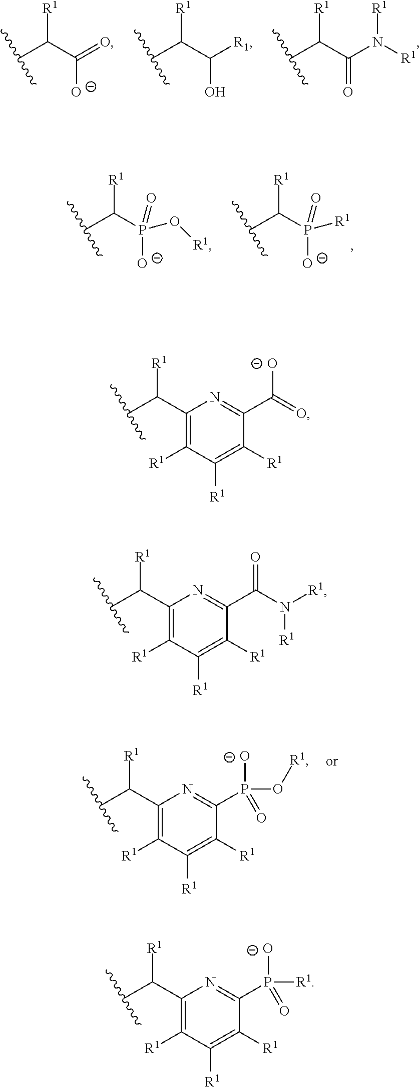

With respect to R², a person of ordinary skill in the art understands that when the ligand is not coordinated to a metal in a CA, any atom shown with a charge, such as O—, will either be protonated, such as in —OH, or will have a suitable counter ion, such as a sodium, potassium, calcium, lithium, or magnesium, or a suitable non-metallic ion, such as ammonium. However, in embodiments where the ligand is coordinated to a metal in a CA, the charged atoms form bonds with the metal in the CA.

L is a bond or a linker.

BG is a bioorthogonal reaction pair moiety, such as, but not limited to, a bioorthogonal reaction pair moiety as described herein, for example, in FIGS. 2 and 22. In some embodiments, L is a bond, but in other embodiments L is a linker moiety, and may be —C₁₋₆alkyl-, —C₁₋₆alkyl-C(O)—, —O—C₁₋₆alkyl-, —O—C₁₋₆alkyl-C(O)—, —NHC₁₋₆alkyl-, or —NHC₁₋₆alkylC(O)—, or an aromatic linker, such as -phenyl- or —O-phenyl-, and which may be ortho, meta or para substituted.

In some embodiments, BG is selected from

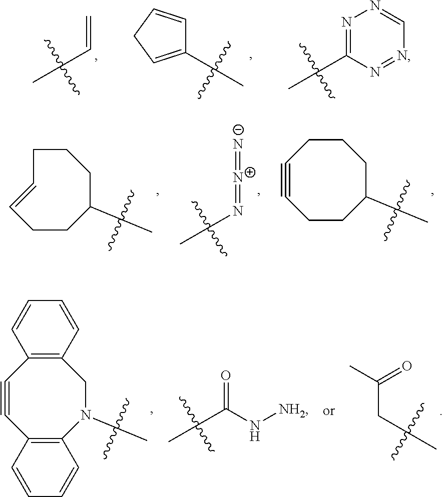

Typically, the CA will further comprise a metal ion suitable for use in a CA. In some embodiments, the metal is Gd³⁺, Mn²⁺ or Fe³⁺, and in particular embodiments, the metal is Gd³⁺

In some embodiments, the CA has a structure selected from the following formulas:

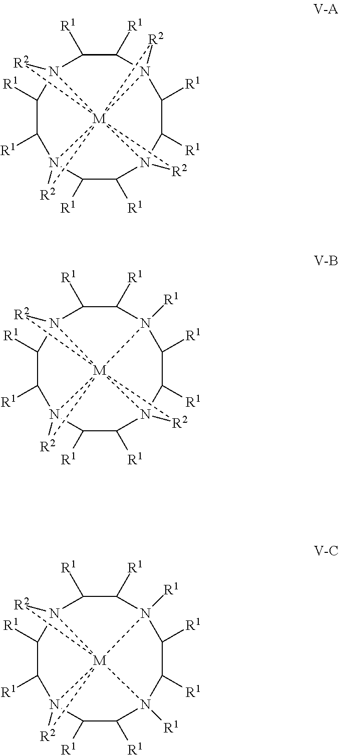

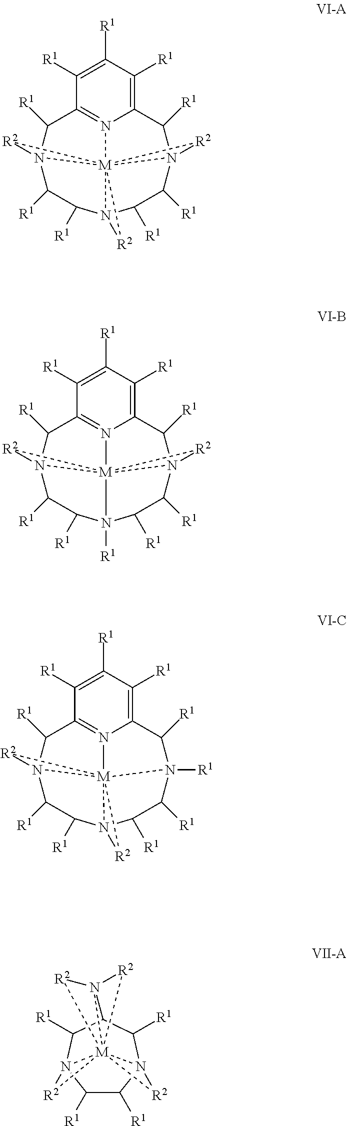

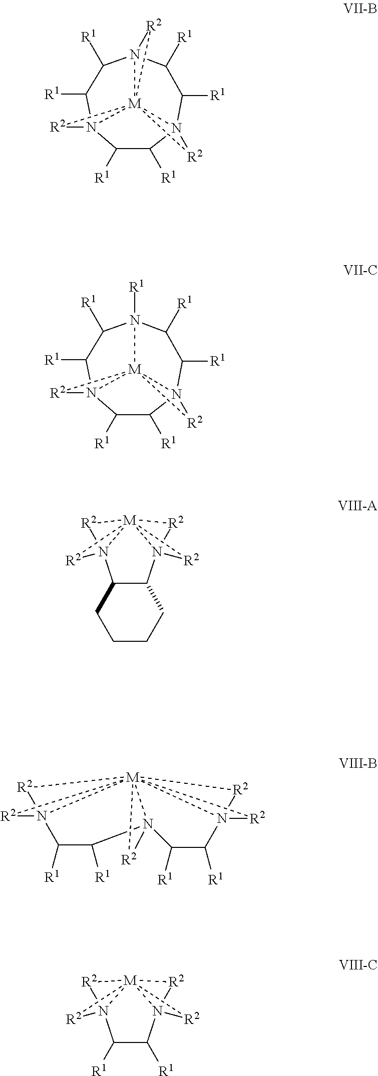

With Respect to Formulas V-A to VIII-C, R¹ and R² are as previously defined for Formulas I-A to IV-C. And M is a metal ion suitable for use in a CA. In some embodiments, M is Gd³⁺, Mn²⁺ or Fe³⁺, and in particular embodiments, M is Gd³⁺.

Also with respect to Formulas V-A to VIIL-C, the dotted and dashed lines indicate optional bonds to the metal M. Specifically, dotted lines “ [symbol] ” indicate an optional bond from the metal M to a nitrogen atom, and dashed lines “ [symbol] ” indicate one or two optional bonds from the [symbol] metal M to an R² substituent. Although the formulas herein show a single dashed line from the R² substituents to M, a person of ordinary skill in the art understands that the dashed line indicates that if a bond exists between the R² substituent and M, the R² substituent may provide one or two bonds between the metal and the R² substituent, depending on the structure of R², such as when the R² substituent comprises more than one oxygen, nitrogen, or a combination thereof. For example, in the exemplary R² moieties provided herein, examples where R² comprises a pyridinyl moiety as well as a carboxylate, amide, or P═O structure may provide one or two optional bonds to the metal in any formula.

With respect to Formulas V-A, V-B, V-C, VI-A, VI-B and VI-C, in some embodiments, at least three of the dotted bonds between the metal and a nitrogen are present, and at least two of the dashed bonds between the metal and an R² substituent are present.

With respect to Formulas VII-B and VII-C, in some embodiments, at least three dotted bonds between the metal and a nitrogen are present. Or in alternative embodiments, the contrast agent has a structure according to Formula VII-B, M is Fe³⁺ or Mn²⁺, and the macrocycle acts as a scaffold where no dotted bonds between the metal and nitrogen are present, but each of the dashed bonds between the metal and the R² substituents are present. And in certain embodiments, one or more R² substituent may provide two bonds between the metal and the R² substituent, such as when the R² substituent comprises more than one oxygen, nitrogen, or a combination thereof.

In any embodiments of the Formulas V-A to VIII-C, the CA comprises at least three of the optional dotted and/or dashed bonds, such as at least four of the optional bonds, at least five of the optional bonds, at least six of the optional bonds, or at least seven of the optional bonds to the metal are present.

In certain embodiments, M is Gd³⁺ and the CA comprises at least seven of the optional bonds to the metal.

In other certain embodiments, M is Fe³⁺ or Mn²⁺, and the CA comprises at least five of the optional bonds to the metal.

In particular embodiments, the CA has a structure according to Formula IX:

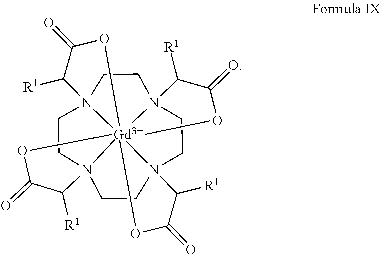

With respect to Formula IX, each R¹ independently is R or —L—BG, where at least one R¹ is —L—BG.

In some embodiments, exactly one R¹ is —L—BG.
M is as defined for Formulas V-A to VIII-C.
Each R, L and BG independently is as defined for Formulas I-A to IV-C.
In other particular embodiments, the CA has a structure according to Formula X:

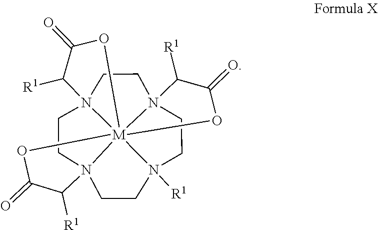

With respect to Formula X, each R¹ independently is R or —L—BG, where at least one R¹ is —L—BG.

In some embodiments, exactly one R¹ is —L—BG.
M is as defined for Formulas V-A to VIII-C. In certain embodiments of Formula X, M is Mn²⁺ or Fe³⁺.
Each R, L and BG independently is as defined for Formulas I-A to IV-C.
Exemplary compounds according to the disclosed formulas include, but are not limited to:

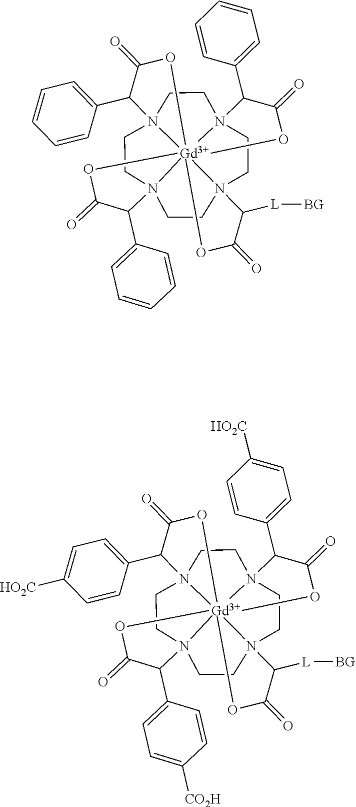

Some embodiments of the disclosed formulas where each R is not H are stereoisomers with stereochemistry at the carbon atoms bonded to R (i.e., stereochemistry at the carbon atoms alpha to the nitrogen atoms). With four stereocenters, RRRR-, SSSS-, RRRS-, SSSR-, RSRS-, and RRSS-isomers are possible. In some embodiments, the compound is a diastereomer, and molecules of the diastereomer comprise a mixture of RRRR- and SSSS-enantiomers. For example, the compound may be:

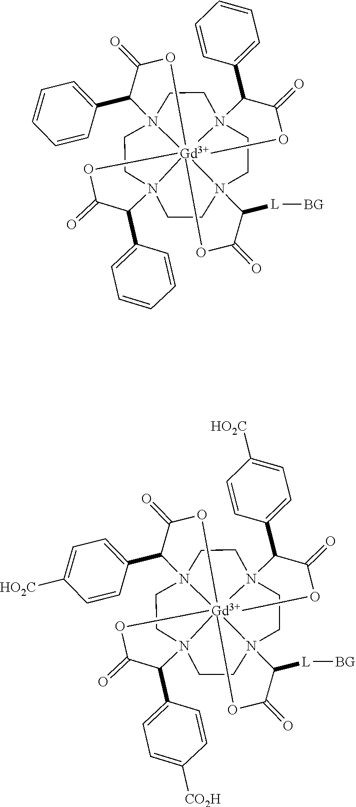

### V. Polystyrene Bead Functionalization and Characterization

### A. Synthesis and Characterization of Azide Terminated Polystyrene Microbeads

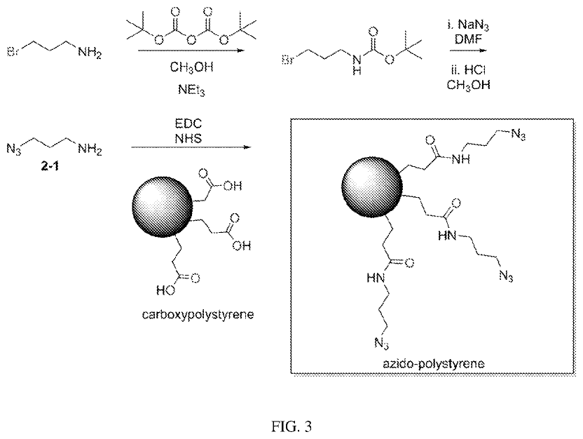

For heterogeneous interception, carboxypolystyrene microbeads (200-400 mesh, 2.06 mM g⁻¹ loading capacity) were purchased from Rapp Polymere to further functionalize with bioorthogonal partners through amide linkages. For the azide functionalization strategy, 3-azidopropan-1-amine was synthesized through a two-step BOC protection of the amine of 3-bromopropan-1-amine, followed by an S_{N}2 substitution of the alkyl bromide to introduce the azide, and finally deprotection of the primary amine to generate the desired product 3-azido-propan-1-amine (2-1), shown in FIG. 3. The carboxypolystyrene was subsequently functionalized with 2-1 through an activated carbodiimide intermediate accessed with EDC and NHS.

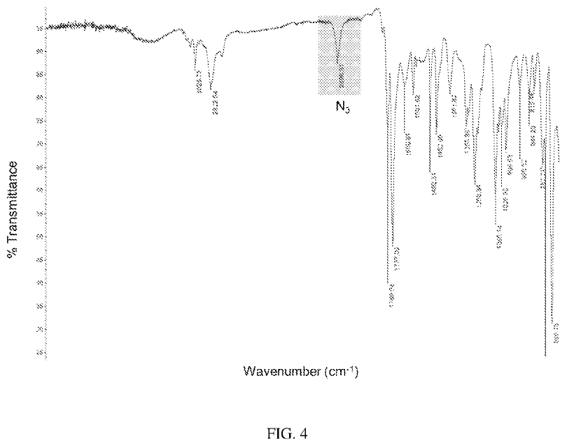

The resulting beads were analyzed by IR for preliminary confirmation of azide functionalization as indicated by a characteristic —N₃ stretch at 2096 cm⁻¹ (FIG. 4). To estimate the loading capacity of the azide terminated PS beads, a fluorescent dye was loaded onto the beads through a Cu(I)-catalyzed click reaction and the immobilized dye was quantified by fluorescence spectroscopy. This analytical method was predicated on the assumption that the fluorescent dye was loaded in high yield. Additionally, the fluorescent beads were undissolved during analysis and each measurement was determined by an average of replicates after agitation. These studies provided a reasonable estimate of the minimum azide loading capacity that were applied to further studies.

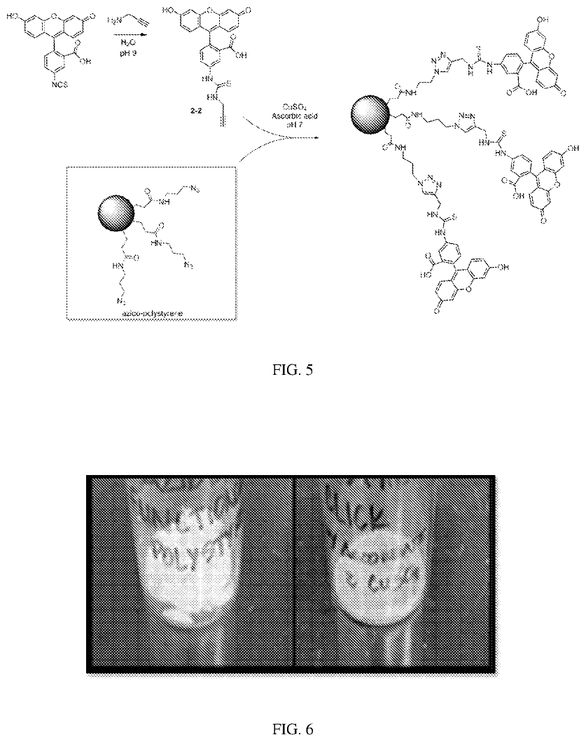

A fluorescein-based dye containing an isothiocyanate group was used that coupled to an amine-containing terminal alkyne through a thiourea linkage, as shown in FIG. 5. The alkyne-functionalized dye (2-2) was purified by preparatory HPLC and confirmed by ESI-MS before undergoing a CuAAC click reaction with the azide terminated beads using CuSO4 copper(II) source and ascorbic acid reducing agent to generate the Cu(I) catalyst in situ (FIG. 5). The resulting beads were washed with acetonitrile and water followed by drying in vacuo overnight. The dried beads were bright orange in color (FIG. 6) which was consistent with the presence of fluorescein dye.

The beads were analyzed by fluorescence to quantify the loading capacity of the dye, which in turn provided an estimate of the azide loading efficiency. Fluorescence studies were carried out by first determining λₘₐₓ over a range of 500 nm-710 nm with excitation at 495 nm. The maximum emission appeared at 519 nm and was used to generate an external calibration curve of FITC dye ranging from 0-1 μM. The FITC-PS functionalized beads were measured and the loading capacity was calculated as a function of the external calibration curve. The calculated loading capacity was found to be 0.2885 μM g⁻¹, a 2.64% yield.

### B. Synthesis and Characterization of DBCO-Polystyrene Microbeads

In initial attempts at DBCO functionalization of the carboxypolystyrene beads, a reported literature synthesis for activated coupling was followed. The beads were mixed overnight in a solution of EDC, NHS and MES buffer (pH 6) followed by centrifugation and removal of the supernatant. The beads were then washed with water and centrifuged (×3) before analysis by IR spectroscopy and UV-Vis absorbance. For IR analysis, it was expected that successful functionalization would result in C—C and aromatic C—H stretches at 2100 cm⁻¹ and 3300 cm⁻¹, respectively. For UV-Vis analysis, λₘₐₓ of the DBCO amine starting material was identified at 309 nm to test for incorporation by absorbance. However, neither method provided evidence of successful functionalization. Without being bound to a particular theory, a deleterious ester hydrolysis may have taken place which halted the reaction. Accordingly, the synthesis was carried out in THE under inert atmosphere to limit access of water. After stirring overnight, the beads were once again centrifuged, washed three times with acetonitrile and dried in vacuo. Unfortunately, the IR and UV-Vis absorbances once again did not show evidence of DBCO incorporation.

Activated coupling was also attempted using HBTU coupling reagent with diisopropylethylamine in dry DMF under inert atmosphere following a literature method that has been utilized in coupling DBCO to biomolecules. After stirring overnight, the beads were once again analyzed by IR and UV-Vis absorbance, yet the synthesis was once again unsuccessful. This synthesis was repeated substituting diisopropylethylamine with inorganic K₂CO₃, again without success.

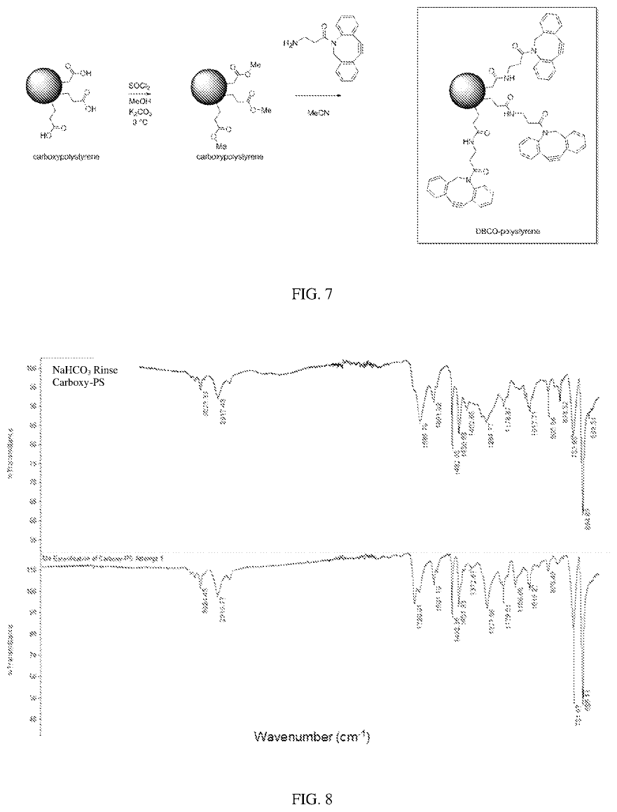

Finally, a two-step functionalization strategy was used, instead of an activated coupling approach. Namely, the carboxypolystyrene beads were first esterified with a methyl ester which should undergo substitution with the corresponding DBCO amine to generate an amide linkage (FIG. 7).

The carboxypolystyrene beads were suspended in MeOH with K₂CO₃ and cooled to 0° C. before SOCl₂ was added dropwise. The reaction was left under reflux overnight before concentration in vacuo. The remaining acid was diluted with water before filtration under vacuum. The resulting beads were rinsed, neutralized with NaHCO₃ and left to dry in vacuo over several hours. The beads were then analyzed by IR to determine whether there was a major difference in the spectra of the starting material and initial attempt at methyl esterification. The IR spectra of the starting material and methyl esterified beads were distinct with additional peaks in the esterified spectrum 1100 cm⁻¹ range that could be attributed to successful methyl esterification. However, due to the neutralization of the beads following esterification, it was unclear whether the neutralization itself was the main source of the change in spectra due to altering the bonding nature from COH to CO⁻Na⁺. Therefore, for direct comparison, carboxypolystyrene bead starting material was neutralized with NaHCO₃ and the spectra were analyzed. As shown in FIG. 8, the spectra were not identical and there were several changes in the peak range 1100-1000 cm⁻¹ that could be attributed to a CO ester stretch. Consistent with direct analyses of the beads, these results strongly indicated methyl esterification.

The methyl esterified beads were suspended in MeOH and CH₃CN before the addition of DBCO amine based on one equivalent of the reported loading capacity of carboxypolystyrene (2.06 mmolg⁻¹). The beads were left to mix overnight at 60° C. before centrifugation and washing with alternating water and CH₃CN to remove excess DBCO amine. The beads were dried in vacuo over several hours before analysis by IR and UV-Vis. The IR spectrum of the resulting beads was not altogether distinct from the starting material; however, there was an additional peak at 1651 cm⁻¹ which was not observed in the methyl esterified bead analysis which it was thought could be indicative of an amide CO stretch. Although the IR spectrum provided vague evidence at best, the UV-Vis absorbance analysis provided significantly more compelling evidence of DBCO incorporation. Namely, the absorbance at 309 nm was significantly increased in comparison to the esterified and carboxypolystyrene bead starting materials. However, when the synthesis was scaled up, there was no evidence of significant incorporation by UV-Vis analysis. Therefore, moving forward the beads were functionalized in small scale batches which then were combined to calculate the average loading capacity through utilization of Beer's Law.

The DBCO-polystyrene microbeads were characterized using the known DBCO molar extinction coefficient of ε=12,000 M⁻¹ cm⁻¹. This was used to determine a loading capacity. For the first batch of beads, the loading capacity was found to be 30.6 μmol g⁻¹. Each batch of beads throughout experiments described herein were on the order of 10 μmol g⁻¹.

### VI. Synthesis of DBCO and Azide-Containing Chelates

Bioorthogonally functionalized Gd-coordinating ligands are commercially available for research purposes, typically used in conjugation to large biostructures. For initial studies a cheaper alternative to the more expensive commercially available ligand precursors and DBCO amine starting material was synthesized. The first studies were carried out with a terminal alkyne-functionalized GdTTHA complex, and further studies were carried out with azide and DBCO functionalized tri-acetate monoamide chelates.

### A. GdTTHA-Alkyne Synthesis

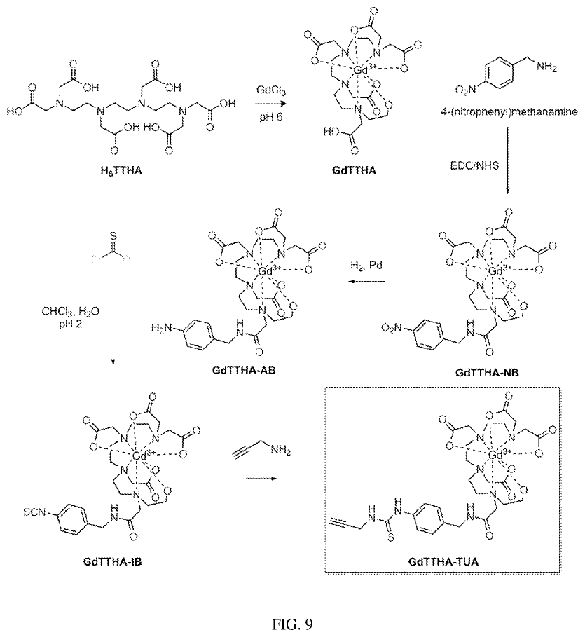

For initial click study experiments, a cheap and synthetically accessible chelate was used, before moving on to more expensive materials. Although the agent would not meet standards for relaxometric efficiency, linearly based TTHA was used that binds Gd in a nine-coordinate fashion with one free carboxylate available for functionalization with a bioorthogonal partner. A potential difficulty in purification without a chromatographic handle was identified and accordingly a nitrobenzyl group was introduced onto the carboxylate that could undergo functional group transformation to the corresponding benzylamine and further conversion to an isothiocyanate upon reaction with thiophosgene. The NCS group was then coupled to prop-2-yn-1-amine through a thiourea linkage to generate the target compound (FIG. 9). The resulting complex was purified by preparatory-HPLC and confirmed by HR-MS before utilization in click trials.

### B. GdDO3A-DBCO Monoamide Synthesis

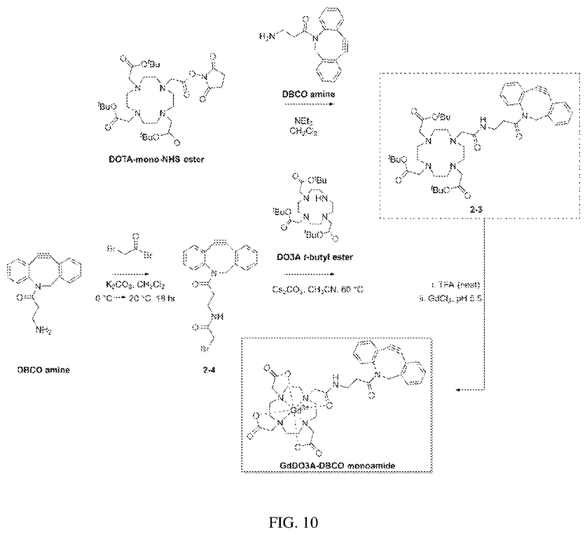

For DBCO incorporation onto a chelate, a mono-amide linkage was selected which was simple to access through coupling DBCO amine with a commercially available mono-NHS ester ligand (FIG. 10 top). The DBCO arm 2-4 was alkylated onto cyclen through standard alkylating methods in CH₃CN with inorganic Cs₂CO₃ base. The esters were cleaved with neat trifluoroacetic acid and the ligand was subsequently chelated with GdCl₃ at pH 5.5. The complex was purified by preparatory HPLC and confirmed by ESI-MS prior to interception studies.

Alternatively, the GdDO3A-DBCO Monoamide may be purchased from Macrocycles, Inc., Plano, TX, USA.

### C. GdDO3A-Azido Monoamide Synthesis

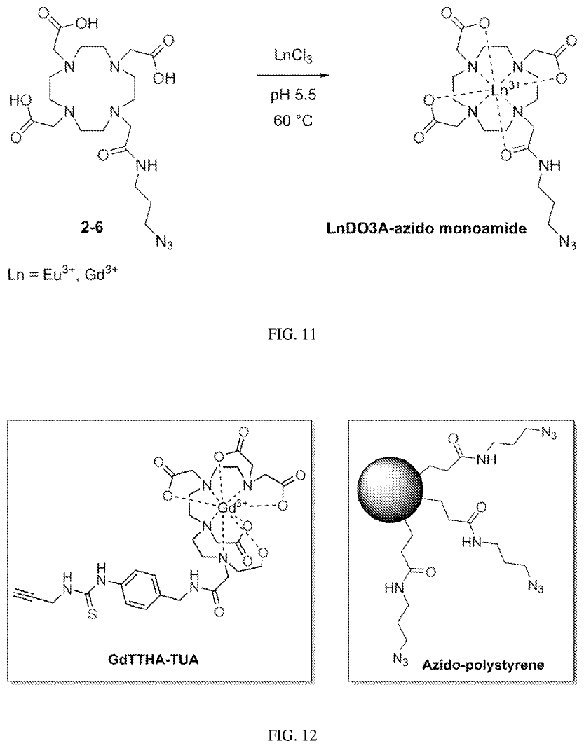

Functionalization with the azide bioorthogonal partner presented a difficult challenge for purification. Initially, coupling the agent through a similar method as the DO3A-DBCO monoamide was considered. However, the complex lacked a chromatographic handle that would allow for preparatory HPLC purification. Therefore, an azide-containing ligand (2-6) was purchased for direct chelation with one equivalent of EuCl₃ and GdCl₃ (FIG. 11). The europium complex was analyzed by ¹H NMR to confirm that chelation was successful, and the gadolinium complex was confirmed by ESI-MS.

Alternatively, the GdDO3A-Azido Monoamide may be purchased from Macrocycles, Inc., Plano, TX, USA.

### VII. Gd Interception Results

A. Preliminary Click Studies with GdTTHA-Alkyne Initial click studies were performed utilizing GdTTHA-Alkyne and azide-PS beads (FIG. 12) on a 0.0265 g and 0.055 g beads scale with 0.5 equivalents of complex in reference to the azide-PS loading capacity as measured through FITC coupling fluorescence studies described herein. The azide terminated beads were suspended in water, followed by the addition of CuSO4 and ascorbic acid reducing agent. The GdTTHA-TUA chelate was subsequently added and the mixture was stirred with a magnetic stirrer bar at 30° C. for 24 hours. ESI-MS was used to analyze the supernatant to test for any remaining chelate and the beads were monitored by IR for a decrease in the N₃ stretch. The resulting mass spectra were somewhat difficult to interpret due to low concentration and a presumably low ionization potential of the complex. However, free ligand was visible in the spectrum after 24 hours, and the azide stretch in the IR spectrum did not appear to change. Ultimately, the trial was considered unsuccessful due to an inadequate analysis technique and the potential of metal dissociation. In addition, it was recognized that mechanical stirring could affect the morphology of the beads. For further studies alternative mixing methods were used. Additionally, a more sensitive analytical technique was used. The supernatant of the reaction was measured by ICP-MS to detect and quantify Gd into the ppb range.

B. SPAAC Interception Studies with DBCO Chelate and Azide PS

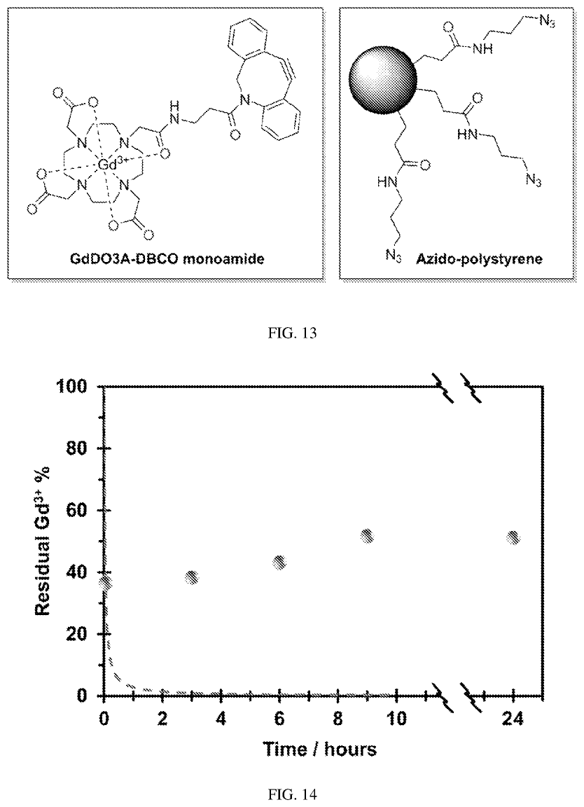

To address potential stability issues associated with the GdTTHA-Alkyne and azide-PS beads system, tri-acetate mono-amide chelate systems were tested, that may experience fewer experimental difficulties. The initial SPAAC reactions were carried out with GdDO3A-DBCO monoamide chelate and azide terminated bead (FIG. 13). The Gd³⁺ concentration was temporally monitored in the supernatant to gain a sense of reaction kinetics and optimal reaction times. To avoid mechanical stirring, a temperature-controlled thermomixer was used which mixed through agitation and was capable of batch testing. And the reaction was analyzed by ICP-MS.

All experiments were performed on a 100 mg bead scale with 0.5 equivalents of GdDO3A-DBCO monoamide chelate in water (1 mL) agitated at 1,000 rpm and 50° C. Four time points were selected: 3 minutes, 3 hours, 6 hours, 9 hours and 24 hours. The reactions were stopped at their respective time point, the sample was centrifuged and the supernatant was decanted and filtered. The beads were washed and centrifuged three times before the filtered supernatant was freeze dried and digested in HNO₃ for ICP-MS analysis.

The initial data obtained from ICP-MS indicated by blue spheres revealed interesting and unexpected results (FIG. 14). The percentage of Gd that was removed from the reaction mixture after three minutes was promising at over 60% removal. To ensure that 60% removal after three minutes was consistent with the reported rates of reaction, the integrated second order rate law was plotted and the rate constant was extrapolated as 48.4 M⁻¹ s⁻¹, indicated by the red dashed line in FIG. 14. The calculated rate constant fell within the literature ranges of 1-60 M⁻¹ s⁻¹.

However, the percentage of Gd removed appeared to decrease over time before leveling off after 24 hours. Unsure of what could be causing the reversal of the trend, the pH of the stock solution was tested and found to be quite low at pH 2. It was hypothesized that although 60% of the chelate had undergone interception onto the polystyrene beads, the metal was subsequently liberated from the complex back into solution by acid-catalyzed hydrolysis. Accordingly, the pH of the stock solution was raised, and further experiments were pH-controlled to ensure metal hydrolysis was prevented.

### i) Variant pH and Cosolvent Tests

The effect of pH at relevant pH ranges of urine (~4.5-8) on the efficiency of Gd removal was investigated. Additionally, the role of an organic cosolvent was investigated to determine whether the aqueous medium is a limiting factor in efficiency. A pH range of 5-9 was tested with 1:1 v:v DMSO:H₂O at 50° C. stirring at 1,000 rpm for 24 hours. The reaction mixtures were centrifuged, and the supernatant was decanted and filtered. The beads were washed and centrifuged in triplicate and the supernatants were freeze dried and digested for ICP-MS analysis, data summarized below in Table 1.

TABLE 1
Data from the pH experiments

| pH | % Gd Remaining |
|---|---|
| 5 | 1.64 |
| 7 | 2 |
| 9 | 1.6 |

The efficiency of Gd removal in these experiments was significantly increased, approaching 100%. The dependence on pH did not appear to play a significant role after 24 hours above pH 5, suggesting that the organic cosolvent played a contributing role in promoting the reaction to reach near completion. Without being bound to a particular theory, the hydrophobicity of the bulky aromatic DBCO moiety may have led to intermolecular self-association of the chelate, limiting access of the bead surface for electrocyclization to occur. Introduction of an organic solvent may have aided to lift the self-association and promote higher reaction efficiency.

### ii) Cosolvent, Reaction Time and Temperature Studies

A multivariate approach was designed to establish the interaction between various levels of cosolvent volume, reaction time and temperature. The volume of DMSO was lowered with the hope of identifying other contributing factors that would lead to an enhanced outcome using a minimal amount of organic cosolvent. Additionally, the temperature ranged from 25° C. to 50° C. and the reaction time was assessed over 0.5 hours and 24 hours, and at pH 7. The scale of the beads and GdDO3A-DBCO monoamide chelate remained the same as previous trials at a 100 mg scale with 0.5 equivalents of GBCA while mixing at 1,000 rpm. The beads were centrifuged, washed, and digested in nitric acid for ICP-MS analysis using the methods described previously, results summarized below in Table 2.

TABLE 2
Results from multivariate optimization study of GdDO3A-
DBCO monoamide and azide terminated PS beads.

| | Cosolvent | Temperature | Reaction Time | Gd Remaining |
|---|---|---|---|---|
| Entry | Volume (μL) | (° C.) | (hr) | (%) |
| 1 | 0 | 25 | 0.5 | 71 |
| 2 | 0 | 25 | 24 | 70 |
| 3 | 100 | 25 | 0.5 | 68 |
| 4 | 100 | 25 | 24 | 69 |
| 5 | 0 | 50 | 0.5 | 67 |
| 6 | 0 | 50 | 24 | 65 |
| 7 | 100 | 50 | 0.5 | 69 |
| 8 | 100 | 50 | 24 | 67 |

The results from these studies were not expected. The efficiency of removal was in the 30% range with little variability or interactions between variables. Because this was a significant decrease in efficiency from the previous study, it was hypothesized that the minimal amount of DMSO may not be sufficient to overcome the supposed self-association of the DBCO-containing chelate. Additionally, samples for ICP-MS analysis were prepared at a higher concentration with fewer dilutions to try to reduce errors being introduced at that stage.

iii) Cosolvent, Matrix and Time Studies

Following the suboptimal results from previous trials with a smaller volume of DMSO cosolvent, the range of added cosolvent was increased to 50% v:v. Additionally, a simulated urine matrix, Surine™, was added to test if there were any significant effects from any urine constituents on the reaction efficiency. The experiments were run with variable reaction times, matrix and percentage DMSO at pH 7 and 37° C. The results are displayed in Table 3.

TABLE 3
Results from multivariate optimization study of GdDO3A-
DBCO monoamide and azide terminated PS beads

| | Reaction Time | | % DMSO | Gd Remaining |
|---|---|---|---|---|
| Entry | (hr) | Matrix | (v:v) | (%) |
| 1 | 0.5 | Water | 0 | 27 |
| 2 | 0.5 | Water | 50 | 28 |
| 3 | 8 | Water | 0 | 27 |
| 4 | 8 | Water | 50 | 18 |
| 5 | 0.5 | Surine ™ | 0 | 28 |
| 6 | 0.5 | Surine ™ | 50 | 29 |
| 7 | 8 | Surine ™ | 0 | 30 |
| 8 | 8 | Surine ™ | 50 | 20 |

The results from these trials were more encouraging, reaching a maximum of 82% Gd removal. A larger volume of DMSO appeared to impart a relatively high positive effect on the efficiency outcome, which was consistent with the previous hypothesis concerning the organic cosolvent disrupting self-association of the chelate. The reaction time also influenced the efficiency of interception with a positive effect for longer reaction times, which was consistent with initial successful trials done over 24 hours. The interaction between the % DMSO and reaction time likewise had positive effect. In the Surine™ matrix studies, the same trends were observed and the average Gd removal percentages of the two studies were quite similar. Therefore, these results suggested that the Surine™ matrix did not negatively influence the reaction efficiency significantly, further validating proof of principle for selective interception from a urine matrix. To understand the interplay of functionalization strategy, these experiments were repeated using the GdDO3A-azido monoamide chelate and DBCO-PS beads.

C. SPAAC Interception Studies with Azide Chelate and DBCO Beads

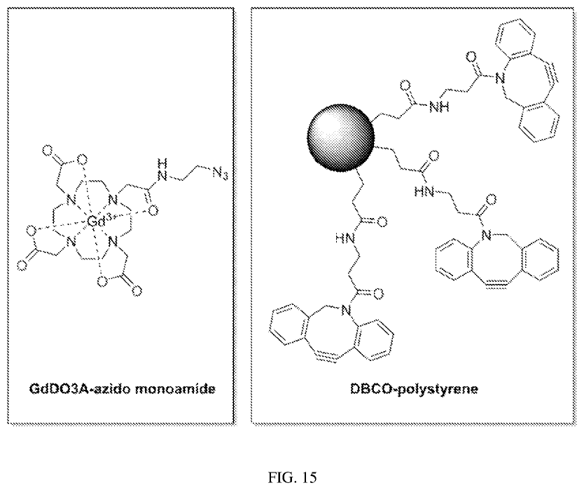

The GdDO3A-azido monoamide chelate and DBCO-PS beads shown in FIG. 15 were utilized in a similar experiment described previously. Due to the expensive DBCO starting material, the beads were generated on small scales. Accordingly, the scales of the experiments were reduced to 50 mg bead starting material and 0.5 equivalents of GBCA consistent with the loading capacity determined for each batch of DBCO-PS. All other conditions remained constant (1 mL reaction volume, 37° C., 1,000 rpm mixing at pH 7). Samples were prepared for ICP-MS analysis using the same method as previously described with fewer dilutions. The experiments were run with variable reaction times, matrix, and percentage DMSO. The results are displayed in Table 4.

TABLE 4
Results from multivariate optimization studies of
GdDO3A-azido monoamide and DBCO terminated PS beads
with variable reaction time, matrix and % DMSO.

| | Reaction Time | | % DMSO | Gd Remaining |
|---|---|---|---|---|
| Entry | (hr) | Matrix | (v:v) | (%) |
| 1 | 0.5 | Water | 0 | 39 |
| 2 | 0.5 | Water | 50 | 36 |
| 3 | 8 | Water | 0 | 40 |
| 4 | 8 | Water | 50 | 35 |
| 5 | 0.5 | Surine ™ | 0 | 27 |
| 6 | 0.5 | Surine ™ | 50 | 37 |
| 7 | 8 | Surine ™ | 0 | 39 |
| 8 | 8 | Surine ™ | 50 | 38 |

The results shown in Table 4, from the GdDO3A-azido monoamide chelate and DBCO-PS beads, suggested a comparable yet slightly less efficient system in comparison to the GdDO3A-DBCO monoamide studies with a maximum of 73% Gd removal. Unexpectedly, the influence of DMSO cosolvent observed in the water matrix imparted a positive effect on the outcome with larger volume of organic cosolvent, consistent with the DBCO counterpart. However, it is conceivable that the beads also exhibited some effect of aggregation due to the hydrophobic DBCO moiety. The time of the reaction did not appear to influence the outcome, and there was a small positive impact with a large volume of DMSO in conjunction with longer reaction times. For the Surine™ studies, there appeared to be a relatively large negative impact of DMSO, likely owed to the outlying data point of 27% in the Entry 5 experiment with no added DMSO and 0.5 hour reaction time. Accordingly, there appeared to be a strong influence on efficiency by reducing the reaction time. There was also a relatively high positive impact on the yield as both DMSO volume and reaction time were increased. The results from these studies indicated a relatively high efficiency of Gd interception. The range of variability of Gd interception was low, and it is possible that the variables do not play a significant role in overall efficiency. It is also conceivable that the smaller scale of these experiments led to a less efficient system than the reversely functionalized counterpart.

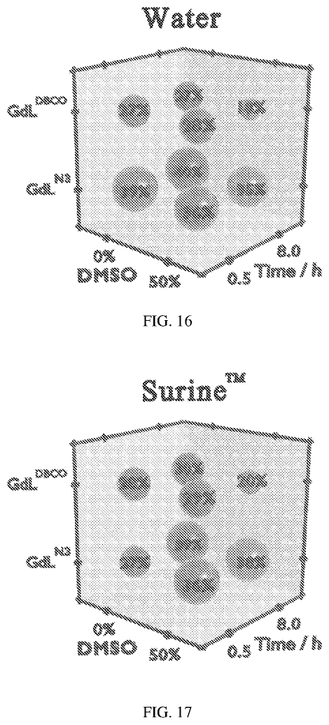

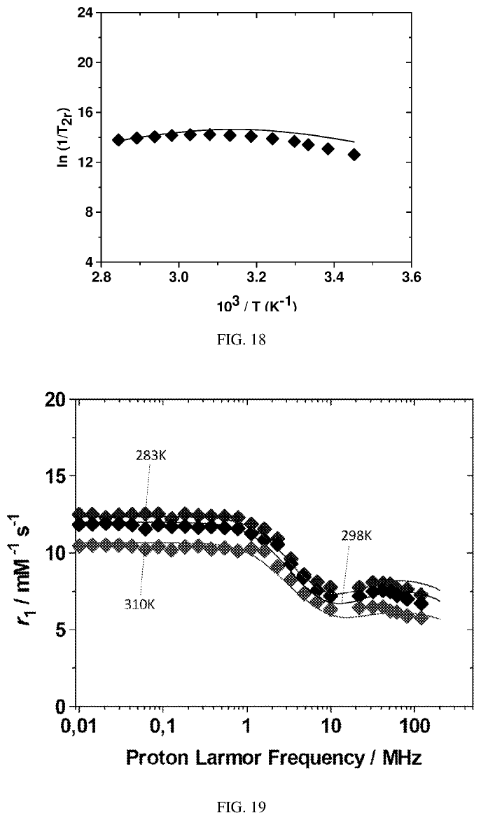

A compilation of the datasets from each functionalization strategy are displayed in FIGS. 16 and 17 to visualize the effects of each variable on the reaction efficiency in water (FIG. 16) and Surine™ (FIG. 17) matrices. As shown below, there did not appear to be a large impact on functionalization strategy in the water matrix. Conversely, on average the GdDO3A-azido monoamide chelate did not perform as well as the DBCO counterpart in the urine matrix. Presumably the Surine™ matrix disrupted the positive effect of cosolvent volume observed in other trials. However, the question remains whether the hydrophobicity of the DBCO moiety was the root cause for the differences in efficiency, if there was an effect borne by scale, or a combination of both. Regardless, each system provided proof of principle for a relatively efficient filtration of GBCAs from Surine™ and water.

VIII. Relaxometric Characterization of GdDO3A-DBCO monoamide and GdDO3A-azido monoamide: Implications for Self-Association

Previous work by the inventors has shown that chelates functionalized with bulky aromatic groups have the propensity to self-associate in solution. Disparities in self-association of two regioisomeric GdNB-DOTA complexes revealed a large difference in the physico-chemical parameters that influence relaxivity. The most common methods to determine the physico-chemical properties described by SBM theory are through fittings of experimental data obtained from the reduced ¹⁷O transverse relaxation rate, R₂ₚ, as a function of temperature and relaxivity, rᵢ, as a function of proton Larmor frequency i.e. nuclear magnetic resonance dispersion (NMRD) profiles.

To probe whether self-association was occurring, the monoamide chelates herein were characterized.

A. GdDO3A-DBCO monoamide Characterization

Analysis of the reduced transverse relaxation rate constant of ¹⁷O (R₂ₚ) as a function of temperature is the most common experimental technique to determine water residence lifetime, τ_{M}. The function typically resembles a bell curve where the inflection point temperature is an indicator of water exchange kinetics. The temperature-dependent R₂ₚ relaxation rate of ¹⁷O are shown in FIG. 17 (top). The experiments were performed at a pH of 6.8 and a Gd³⁺ concentration of 6.5 mM. Precipitation was observed below 295 K. The ¹⁷O curve reached an inflection point at about 310 K, and the fitted value of τ_{M} was 545 ns, comparatively slow in the context of achieving high relaxivities but fast enough that exchange is in the fast exchange regime and rₗ is sensitive to changes in molecular tumbling, τ_{M}.

The NMRD profiles of GBCAs are used to characterize several important physico-chemical parameters described by SBM theory. The most influential parameter which affects the shape of an NMRD curve is the rotational correlation time constant (τ_{R}) of the complex. Macromolecular species which tumble slowly impart a positive impact on relaxivity at all fields, but particularly at high fields presented as a characteristic “hump” in the 10-60 MHz region, distinct from quickly tumbling chelates in which relaxivity continues to decrease with increasing field strength. The molecular weight of the GdDO3A-DBCO monoamide would not be considered a large macromolecular structure; any indication of a slowly tumbling complex would be indicative of an aggregate species which we hypothesized as the self-associating chelate. As shown below in FIG. 19, relaxivity is quite high in low field regions where electronic spin relaxation contributes, followed by a characteristic dispersion around 1 MHz when the contribution drops off. There is a “hump” around 20 MHz which is indicative of a slowly tumbling species, consistent with a self-associating chelate. This macromolecular structure is the source of a rather high relaxivity of 7.1 mM⁻¹s⁻¹ (20 MHz, pH 6.8, 298 K) in comparison to most clinical agents which remain in the 4 mM⁻¹ s⁻¹-5 mM⁻¹s⁻¹ range.

The parametric fitting of these data was more accurate using the Lipari-Szabo approach where contributions from both local and global rotation from a macromolecular structure are incorporated into the spectral density function. The global and local rotational correlation time constants were 375 ps and 107 ps, respectively. These data suggested that there was in fact a larger macromolecular aggregate, presumably a dimer, that rotated over 3× more slowly than the discrete chelate. This further corroborated the presence of a self-association which led to a decrease in the efficiency observed in selective interception trials.

### B. GdDO3A-azido Monoamide Characterization

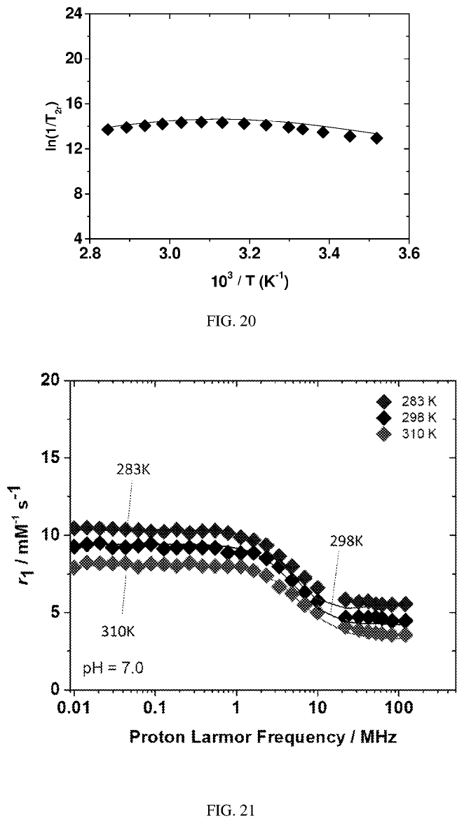

The reduced ¹⁷O transverse relaxation rate as a function of temperature is displayed below in FIG. 20. Unsurprisingly, the shape is nearly identical to the DBCO-containing counterpart; both are monoamides and a similar water exchange rate would be expected. In this case, the water residence lifetime time constant (τ_{M}) was equivalent to the DBCO ligand at 545 ns.

The NMRD profile of the azide-containing chelate is displayed in FIG. 21 and the resulting fittings from both azido- and DBCO-containing chelates are displayed in Table 5. The NMRD profile revealed lower relaxivity across all field strengths in comparison to the DBCO-containing chelate, indicative of a quickly tumbling complex. The dispersion at 1-10 MHz corresponded to a typical drop-off of the electronic spin relaxation contribution, followed by continued decrease in relaxivity with increasing field strength. There was no evidence of a slowly tumbling structure which was reflected in a standard parametric fitting procedure with one discrete rotational contribution. The relaxivity of the azide-containing chelate was less than the DBCO-containing counterpart at 4.7 mM⁻¹s⁻¹ (20 MHz, 298 K, pH 7) due to a shorter τ_{R} However, the relaxivity was comparable to GdDOTA (4.5 mM⁻¹s⁻¹ at 20 MHz and 298 K).

TABLE 5
Calculated relaxometric values fit from ¹⁷O R2p and NMRD
profiles of GdDO3A-DBCO monoamide and GdDO3A-N3-monamide

| | GdDO3A-N₃- | GdDO3A-DBCO- |
|---|---|---|
| | monoamide | monoamide |
| Parameters | pH = 7.0 | pH = 6.8 |
| ²²r₁ (298K) (mM⁻¹ s⁻¹) | 4.7 | 7.1 |
| Δ²/10¹⁹ s⁻² | 5.7 | 3.5 |
| τ_{V}/ps | 8.4 | 13 |
| E_{V}/kJ mol⁻¹ | 1.0^{a} | 1.0^{a} |
| τ_{M}/ns | 545 | 545 |
| Δ H_{M}/kJ mol⁻¹ | 46 | 46 |
| τ_{RG}/ps | 70 | 375 |
| τ_{RL}/ps | / | 107 |
| S^{□} | / | 0.36 |
| E_{R}/kJ mol⁻¹ | 17.9 | 20.0 |
| A_{O}/h/10⁶ rad s⁻¹ | −3.2 | −3.0 |
| q | 1^{a} | 1^{a} |
| r/Å | 3.0^{a} | 3.0^{a} |
| a/Å | 4.0^{a} | 4.0^{a} |
| ²⁹⁸D/10⁵ cm² s⁻¹ | 2.24^{a} | 2.24^{a} |

^{a}Fixed during fitting

The relaxometric characterization supported the hypothesis that the DBCO-containing chelate exhibits self-association. Even though the concentration of the NMRD experiments was 1.32 mM while the interception experiments were more dilute at 0.2 mM, it seems highly likely that aggregation is still occurring to a significant extent in the selective filtration experiments, and corroborated the propensity of the DBCO moiety to undergo intermolecular aggregation in general.

### IX. Conclusion

A system to extract Gd-containing chelates from aqueous media has been developed and initial proof of principle established. The syntheses of several bioorthogonally functionalized Gd-coordinating ligands were carried out and two synthetic routes (GdTTHA-alkyne, GdDO3A-DBCO-monoamide) were described. Carboxypoystyrene beads were functionalized with both azide and DBCO functional groups and their loading capacities were characterized by fluorescence studies and UV-Vis analysis, respectively.

The initial CuAAC click studies with GdTTHA-alkyne and azide PS beads were unsuccessful due to an inadequate analytical technique to quantify Gd interception and the potential for metal hydrolysis. SPAAC bioorthogonal reaction pairs, starting with the GdDO3A-DBCO monoamide and azide terminated PS underwent initial temporal studies. These studies suggested that Gd was liberated from the chelate over time; upon further examination of the reaction mixture, the pH was found to be low and it was hypothesized that the chelate was undergoing acid-catalyzed metal hydrolysis, an unfavorable indication of kinetic inertness. The pH was adjusted for further experiments and an organic cosolvent was added to reach a near quantitative 98% removal of Gd at pHs 5, 7 and 9 with 50% DMSO v:v cosolvent in water. The cosolvent was hypothesized to disrupt any self-aggregation of the hydrophobic DBCO moiety and was further studied at lower volumes without success. The volume of cosolvent was increased to 50% v:v once again while varying reaction time, matrix (Surine™ or water) and % DMSO. The results were encouraging, reaching 80% removal in Surine™ with organic cosolvent and 72% removal in Surine™ without the use of an organic cosolvent.

Reversal of the functionalization on either PS or GBCA was carried out under the same experimental design with varying reaction time, matrix and % DMSO with an azide-containing chelate and DBCO-functionalized PS. The results indicated a less efficient system with a maximum removal of 65% in Surine™ with DMSO cosolvent and 73% without the use of cosolvent. These results indicated that the system may not benefit from the use of an organic cosolvent, presumably due to the lack of aggregation of the complex. In comparison of the two functionalization strategies, it was clear that the functionalization on the complex and bead can significantly affect the efficiency of interception.

Relaxometric characterization of both complexes revealed a slowly tumbling aggregate in the GdDO3A-DBCO monoamide as previously hypothesized. These results supported the initial hypothesis that the organic cosolvent aided in disruption of chelate self-association.

In studies where the functionalization was reversed (DBCO-PS and GdDO3A-N₃ monoamide), the reaction efficiency was reduced overall. Presumably, the variables under investigation did not play a significant role in the outcome of the reaction.

In view of the many possible embodiments to which the principles of the disclosed invention may be applied, it should be recognized that the illustrated embodiments are only preferred examples of the invention and should not be taken as limiting the scope of the invention. Rather, the scope of the invention is defined by the following claims. We therefore claim as our invention all that comes within the scope and spirit of these claims.

1-23. (canceled)

24. A method, comprising contacting a solid support comprising a first bioorthogonal reaction pair moiety with a liquid comprising a contrast agent comprising a second bioorthogonal reaction pair moiety that is complementary to the first bioorthogonal reaction pair moiety.

25. The method of claim 24, wherein the liquid comprises urine or blood that is obtained from a subject who has been administered the contrast agent.

26. The method of claim 24, wherein the contrast agent is a metal chelate comprising a metal ion selected from gadolinium(III) (Gd³⁺), manganese(II) (Mn²⁺) or iron(III) (Fe³⁺).

27. The method of claim 26, wherein the contrast agent comprises a ligand moiety where the ligand moiety is selected from any one of Formulas I-A to IV-C: 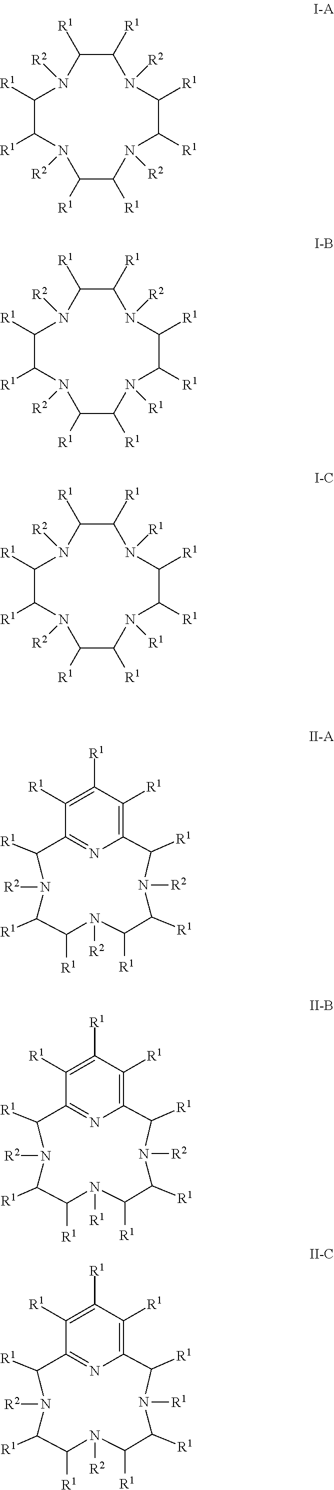 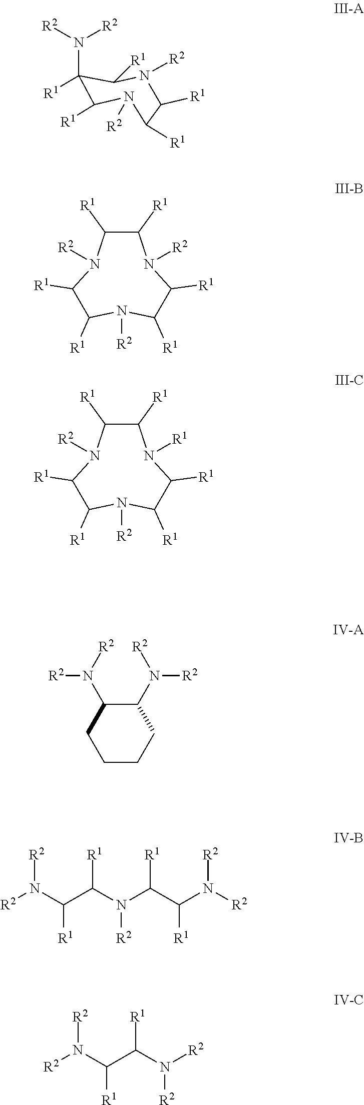
wherein
each R¹ independently is R or —L—BG, where at least one R¹ is —L—BG;
each R independently is H, aliphatic, or aryl;
L is a bond, —C₁₋₆alkyl-, —C₁₋₆alkyl-C(O)—, —O—C₁₋₆alkyl-, —O—C₁₋₆alkyl-C(O)—, —NHC₁₋₆alkyl-, or —NHC₁₋₆alkylC(O)—;
BG is the second bioorthogonal reaction pair moiety; and
R² is selected from

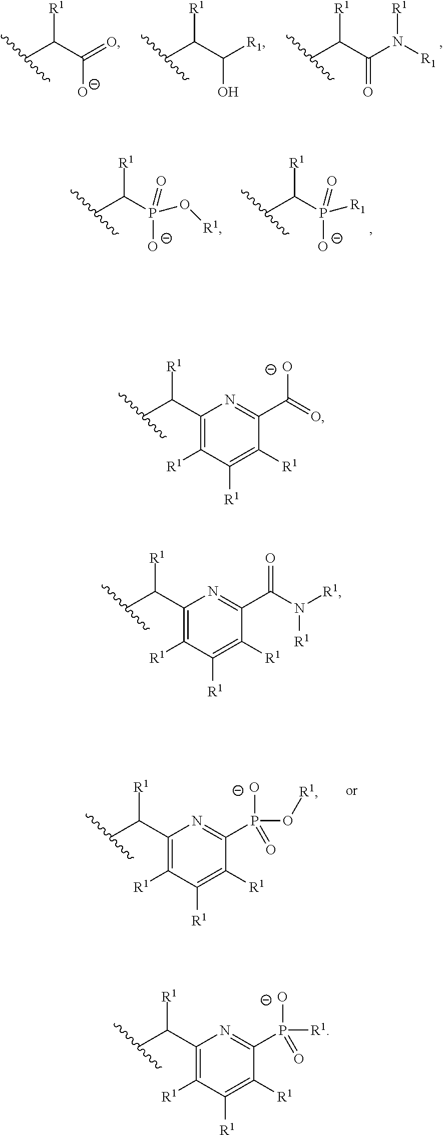

28. The method of claim 27, wherein the contrast agent is a gadolinium-based contrast agent.

29. The method of claim 24, wherein the contrast agent has a structure according to any one of Formulas V-A to VIII-C 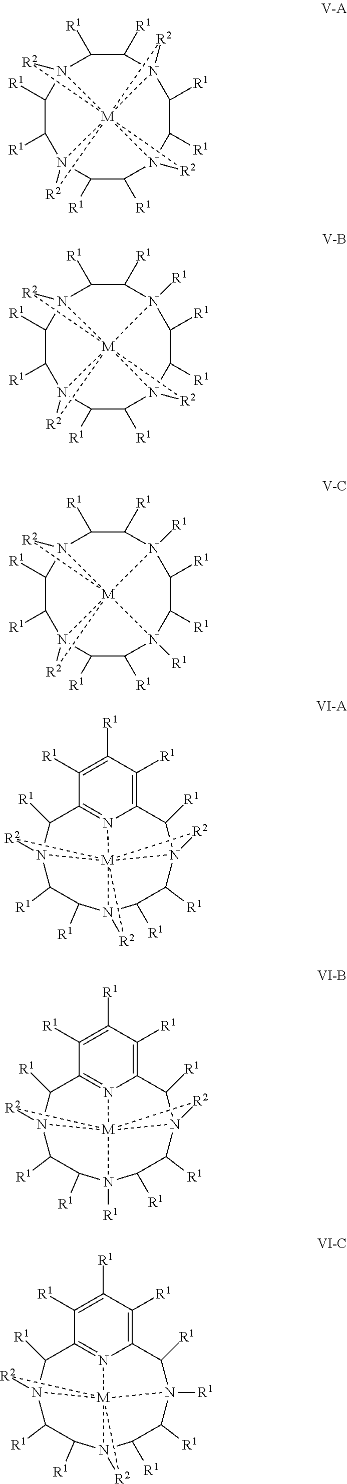 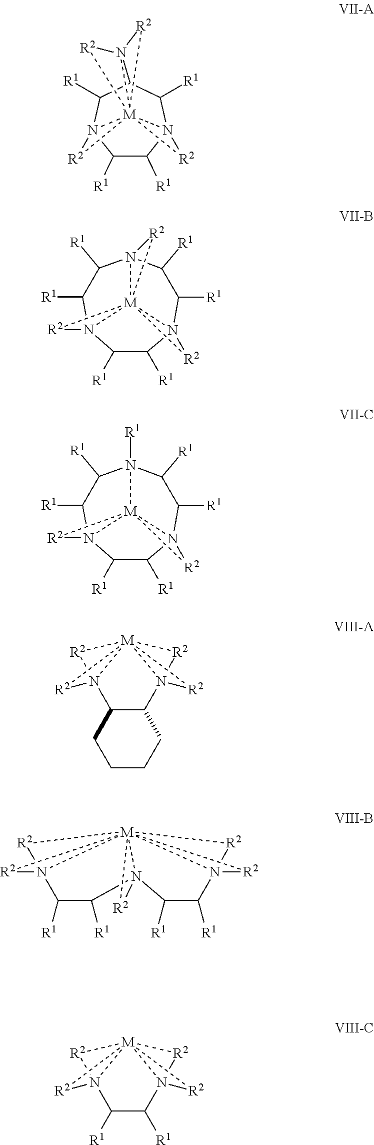
wherein
each R¹ independently is R or —L—BG, where at least one R¹ is —L—BG;
each R independently is H, aliphatic, or aryl;
L is a bond, —C₁₋₆alkyl-, —C₁₋₆alkyl-C(O)—, —O—C₁₋₆alkyl-, —O—C₁₋₆alkyl-C(O)—, —NHC₁₋₆alkyl-, or —NHC₁₋₆alkylC(O)—;
BG is the second bioorthogonal reaction pair moiety;
R² is selected from 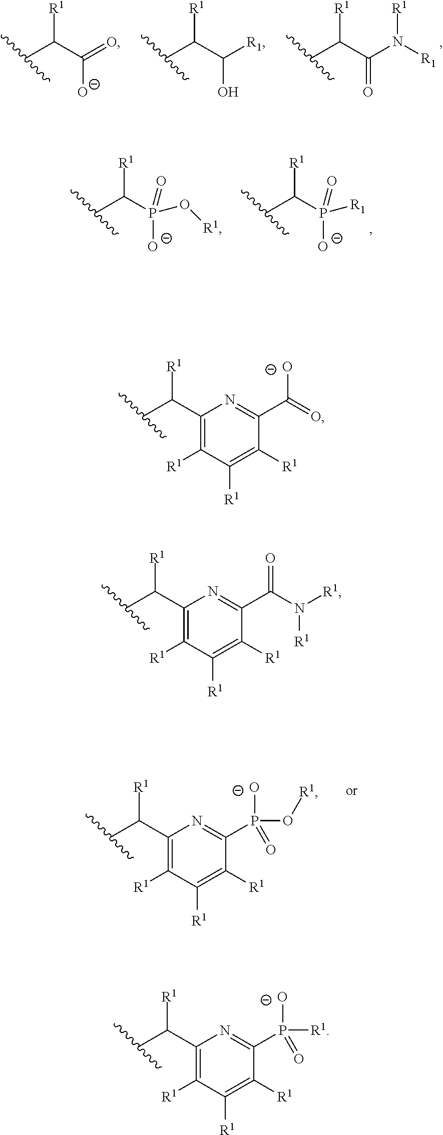
M is selected from Gd³⁺, Mn²⁺ or
dotted lines “ [symbol] ” indicate an optional bond from the metal M to a nitrogen atom;
dashed lines “ [symbol] ” indicate one or two optional bonds from the metal M to an R substituent; and
the contrast agent comprises at least five of the optional dotted and/or dashed bonds.

30. The method of claim 29, wherein:
M is Gd³⁺ and the contrast agent comprises at least seven of the optional dotted and/or dashed bonds to the metal; or
M is Fe³⁺ or Mn²⁺, and the contrast agent comprises at least five of the optional dotted and/or dashed bonds to the metal.

31. The method of claim 29, wherein:
the contrast agent has a structure according to any one of Formulas V-A, V-B, V-C, VI-A, VI-B or VI-C, and at least three of the dotted bonds between the metal and a nitrogen are present, and at least two of the dashed bonds between the metal and an R² substituent are present; or
the contrast agent has a structure according to any one of Formulas VII-B or VII-C, and at least three dotted bonds between the metal and a nitrogen are present.

32. The method of claim 29, wherein the contrast agent has a structure according to Formula VII-B, M is Fe³⁺ or Mn²⁺, and no dotted bonds between the metal and nitrogen are present, but each of the dashed bonds between the metal and the R² substituents are present.

33. The method of claim 24, wherein the contrast agent has a structure according to Formula IX 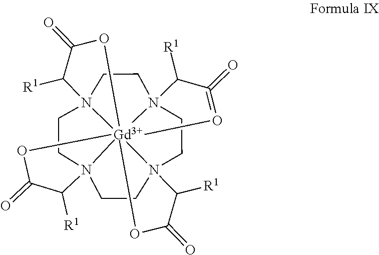
wherein:
each R¹ independently is R or —L—BG, where at least one R¹ is —L—BG;
each R independently is H, aliphatic, or aryl;
L is a bond, —C₁₋₆alkyl-, —C₁₋₆alkyl-C(O)—, —O—C₁₋₆alkyl-, —O—C₁₋₆alkyl-C(O)—, —NHC₁₋₆alkyl-, or —NHC₁₋₆alkylC(O)—; and
BG is the second bioorthogonal reaction pair moiety.

34. The method of claim 24, wherein the contrast agent has a structure according to Formula X 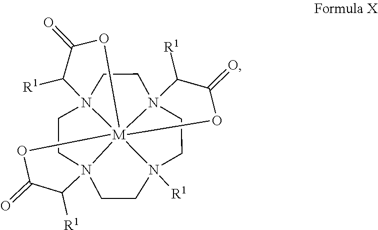
wherein
M is Mn²⁺ or Fe³⁺;
each R¹ independently is R or —L—BG, where at least one R¹ is —L—BG;
each R independently is H, aliphatic, or aryl;
L is a bond, —C₁₋₆alkyl-, —C₁₋₆alkyl-C(O)—, —O—C₁₋₆alkyl-, —O—C₁₋₆alkyl-C(O)—, —NHC₁₋₆alkyl-, or —NHC₁₋₆alkylC(O)—; and
BG is the second bioorthogonal reaction pair moiety.

35. The method of claim 24, wherein the contrast agent has a structure selected from

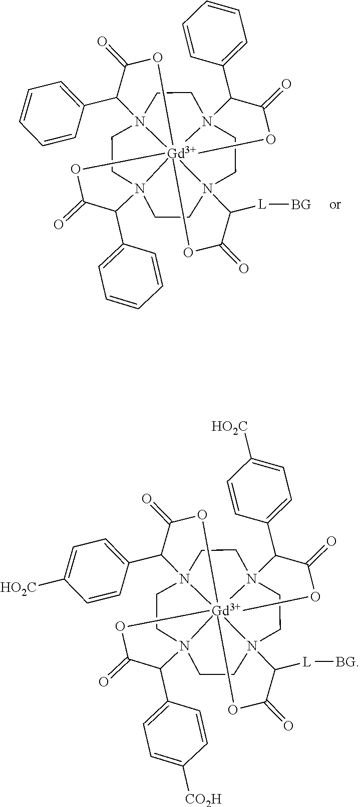

36. The method of claim 24, wherein the first and second bioorthogonal reaction pairs are selected from, in any order:

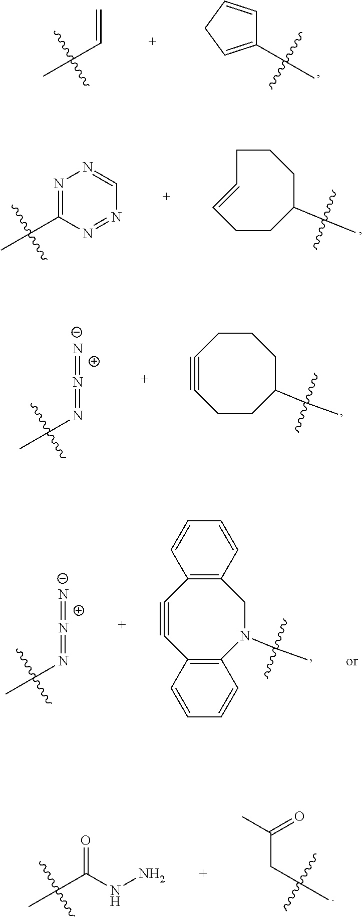

37. The method of claim 24, wherein the solid support comprises silica, peptide resin, or polymeric beads.

38. The method of claim 37, wherein the solid support comprises polystyrene beads.

39. A method, comprising contacting a solid support comprising a first bioorthogonal reaction pair moiety with a liquid comprising a contrast agent comprising a second bioorthogonal reaction pair moiety that is complementary to the first bioorthogonal reaction pair moiety, thereby linking the contrast agent to the solid support; and
separating the contrast agent from the liquid by eluting the liquid from the solid support.

40. The method of claim 39, wherein:
the solid support comprises polystyrene beads;
the contrast agent has a structure selected from 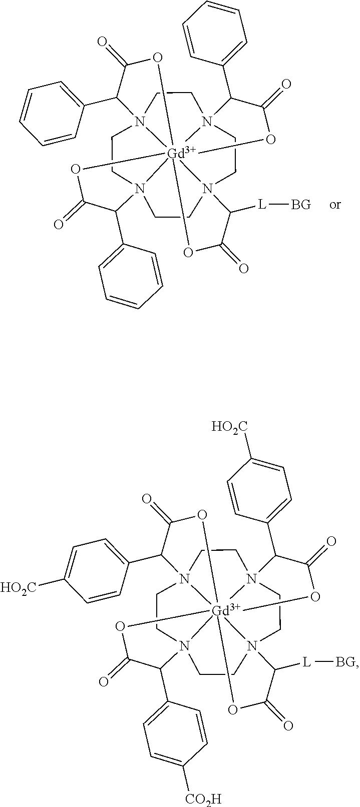
where L is a bond, —C₁₋₆alkyl-, —C₁₋₆alkyl-C(O)—, —O—C₁₋₆alkyl-, —O—C₁₋₆alkyl-C(O)—, —NHC₁₋₆alkyl-, or —NHC₁₋₆alkylC(O)—; and
BG is the second bioorthogonal reaction pair moiety; and
the first and second bioorthogonal reaction pairs are selected from, in any order:

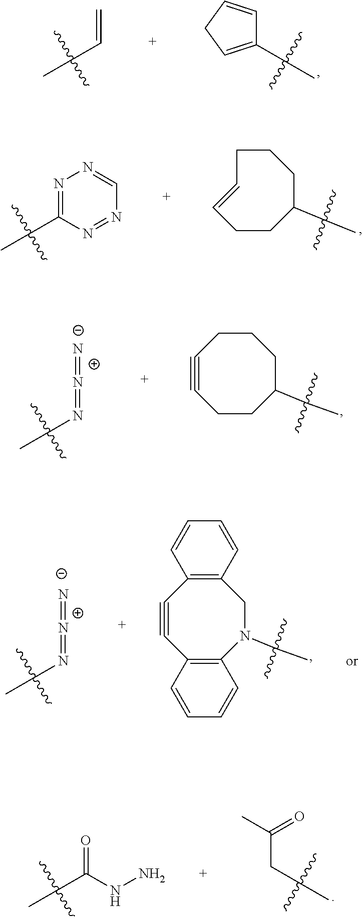

41. A compound having a structure according to any one of Formulas V-A to VIII-C 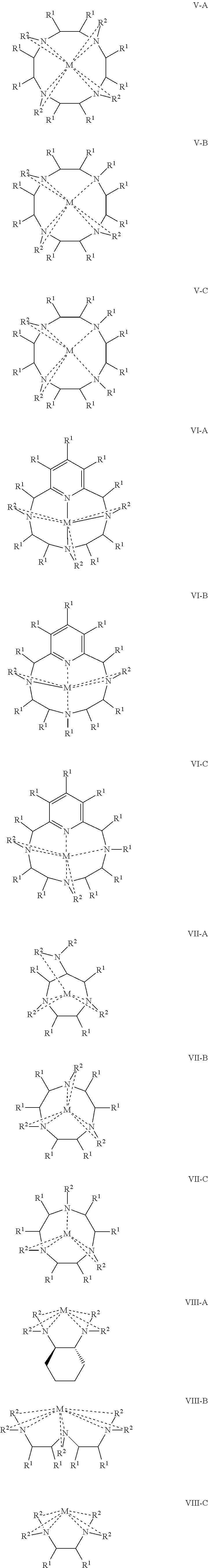
wherein
each R¹ independently is R or —L—BG, where at least one R¹ is —L—BG;
each R independently is H, aliphatic, or aryl;
L is a bond, —C₁₋₆alkyl-, —C₁₋₆alkyl-C(O)—, —O—C₁₋₆alkyl-, —O—C₁₋₆alkyl-C(O)—, —NHC₁₋₆alkyl-, or —NHC₁₋₆alkylC(O)—;
BG is a bioorthogonal reaction pair moiety;
R² is selected from 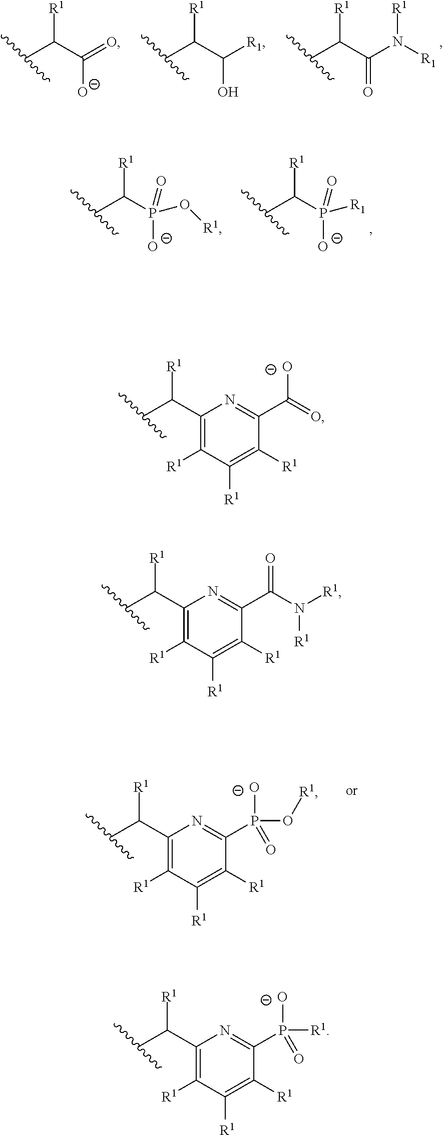
M is selected from Gd³⁺, Mn²⁺ or Fe³⁺
dotted lines “ [symbol] ” indicate an optional bond from the metal M to a nitrogen atom;
dashed lines “ [symbol] ” indicate one or two optional bonds from the metal M to an R² substituent; and
the contrast agent comprises at least five of the optional dotted and/or dashed bonds.

42. The compound of claim 41, wherein BG is selected from:

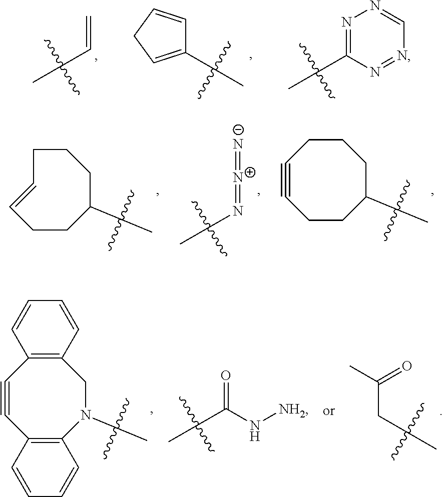

43. The compound of claim 41, wherein the compound is [chemical structure]
